(19) United States
(12) Patent Application Publication
(10) Pub. No.: US 2003/0224422 A1
(43) Pub. Date: Dec. 4, 2003

(54) Pre-and post therapy gene expression profiling to identify drug targets

(75) Inventors: William Edward Evans, Cordova, TN (US); Mary V. Relling, Cordova, TN (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: St. Jude Children's Research Hospital, Inc.

(21) Appl. No.: 10/407,790

(22) Filed: Apr. 4, 2003

Related U.S. Application Data
(60) Provisional application No. 60/370,835, filed on Apr. 8, 2002.
(60) Provisional application No. 60/449,893, filed on Feb. 25, 2003.

(51) Int. Cl. C12Q 1/68

(52) U.S. Cl. 435/6

(57) ABSTRACT

A general method for identifying biological targets for improving currently available therapies is provided. Target genes and their expression products are identified based on their response to therapy as determined through pre- and post-therapy expression profiles. In another aspect, differences in expression profiles between responsive and nonresponsive patients are taken into account to identify potential new targets for the development of novel medications or treatments. The invention also provides methods for comparing therapies to predict which will have the best therapeutic efficacy and/or the least potential deleterious. The methods taught are specifically applied to identify targets for improving treatment of acute lymphoblastic leukemia.

### CROSS-REFERENCE TO RELATED APPLICATIONS

This application claims the benefit of U.S. Provisional Application No. 60/370,835, filed Apr. 8, 2002 and U.S. Provisional Application No. 60/449,893, filed Feb. 25, 2003, each of which is hereby incorporated in its entirety by reference herein.

### FEDERALLY SPONSORED RESEARCH OR DEVELOPMENT

This invention was made in part with U.S. Government support under National Institutes of Health grant nos. R37 CA36401, R01 CA78224, RO1 CA51001 RO1 CA71907, U01 GM61393, U01 GM61394, and Cancer Center Support Grant CA21765. The U.S. Government may have certain rights in this invention.

### FIELD OF THE INVENTION

The present invention relates generally to drug discovery and more specifically to the identification of biological targets for drug intervention to improve current therapies and to methods of predicting the therapeutic efficacy of combination therapies.

### BACKGROUND OF THE INVENTION

Modern drug discovery efforts rely heavily on the screening of compounds for activity against biological targets; proteins (and the genes which encode them) whose presence, absence or abnormal regulation has been associated with a particular disease or condition. Biological targets are used in standard screening assays for drugs to treat their associated condition. Such assays may be designed to identify compounds that can directly interact and modulate the target protein activity or compounds that affect expression of the target protein.

The major limiting step in this process used to be the availability of a sufficient number and variety of compounds to screen for biological activity. With the advent of combinatorial chemistry and accumulation of vast chemical libraries, the actual screening process oftentimes became the rate-limiting step in this process. The development of high-throughput and ultra-high throughput screening assays has largely removed screening itself as a rate limiting step and allowed entire compound libraries to be screened in a relatively short periods.

With these advances in the number of compounds available for screening and the speed with which screening can be accomplished, attention has become focused on new biological targets. Significant effort has been expended on identifying targets for various diseases and a number of approaches have been created to identify such targets. Still, there remains a significant need for methods to identify potential biological targets to treat diseases or improve current therapy and for methods to predict the therapeutic efficacy of new therapies.

### SUMMARY OF THE INVENTION

The present invention provides methods for identifying biological targets for drug screening to improve currently available therapies for any desired condition. The biological targets are identified based on their response to therapy. According to the invention, genes whose expression prior to a selected therapy are found to be significantly different from their expression subsequent to therapy are identified, along with their expression products, as candidate screening targets for modulating drugs which may be used to improve treatment of the condition.

In another aspect, changes in pre-therapy vs. post-therapy gene expression are further associated with response to therapy. According to this aspect, genes whose change in expression before and after therapy are significantly different in those patients which did not respond favorably to therapy compared to patients which did respond favorably are identified, along with their expression products, as screening targets for drugs which may be used to improve treatment of the selected condition.

The present invention also provides methods for comparing therapies and predicting whether a first therapy will have greater therapeutic efficacy than a second therapy. The method comprises determining the expression levels of one or more genes in a sample from patients before and after treatment with the first therapy and the second therapy, where changes in the expression levels of the genes are correlated with a favorable or unfavorable response to therapy. The changes in the expression levels of the genes before and after treatment with the first therapy are then compared with the changes in the expression levels of the genes before and after treatment with the second therapy to predict whether the first therapy will have greater therapeutic efficacy than the second therapy.

In another aspect, the present invention provides methods for predicting whether a first therapy will have greater deleterious effects in a patient than a second therapy. The method comprises determining the expression levels of one or more genes in a sample from patients before and after treatment with the first therapy and the second therapy, where changes in the expression levels of the genes whose expression levels are determined are correlated with deleterious effects of therapy in a patient. The changes in the expression levels of the genes before and after treatment with the first therapy is then compared with the changes in the expression levels of the genes before and after treatment with the second therapy to predict whether the first therapy will have greater deleterious effects in a patient than the second therapy.

The methods of the invention have been applied to acute lymphoblastic leukemia (ALL) to identify candidate targets for improving currently available therapies. Drug screening using the candidate target genes identified through practice of these methods, along with their expression products, represent a further aspect of the invention.

### DESCRIPTION OF THE FIGURES

FIGS. 1A and 1B schematic representation of the process described in Example 1 to obtain pre- and post treatment gene expression data from acute lymphoblastic leukemia (ALL) patients.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention utilizes gene expression profiling in a unique way to identify genes and their expression products as biological targets for drug intervention to improve currently available therapies. This approach comprises two basic measurements:

1) determining the expression level of one or more genes in a sample from a patient affected by a selected condition prior to treatment with an available therapy; and

2) determining the expression level of the same genes in a corresponding sample following treatment with the therapy.

These measurements can then be compared to determine the effect the therapy has upon the expression of a particular gene. Those genes whose expression is not significantly affected by therapy are excluded as candidate targets for screening. Those genes whose expression is significantly increased or significantly decreased after therapy are identified as candidate targets for drug screening, along with their expression products. Such expression products include RNA and protein products naturally expressed from the subject gene.

The identified candidate targets may then be prioritized according to their attractiveness as screening targets. This assessment can be based on the identity of the target and its function, if known. Targets which have a known and easily assayable function, such as a kinase, a phosphatase, receptors (G-protein coupled receptors, cytokine receptors, etc), apoptotic proteins, hydroxylation, oxidation, conjugation and other enzyme reactions, protein-protein or protein-DNA or RNA interactions, and a series of others will generally be preferred for screening relative to targets which have no known function or whose function is not easily assayable. Targets which are found to play a role in biological pathways known to be directly affected by the subject condition will be particularly preferred.

The methods of the present invention may be applied to any condition where there is an available therapy for which improvement is needed. This includes, but is not limited to, cancers, genetic disorders, infectious diseases, hematological disorders, cardiovascular diseases, dermatological diseases, endocrine diseases, gastrointestinal disorders, etc.

In some embodiments, the present invention provides methods for comparing therapies and predicting whether a first therapy will have greater therapeutic efficacy or greater deleterious effects in a patient than a second therapy. The method comprises determining the expression levels of one or more genes in a sample from patients before and after treatment with the first therapy and the second therapy, where changes in the expression levels of the genes are correlated with therapeutic effects or deleterious effects of therapy in a patient. The changes in the expression levels of the genes before and after treatment for the first and second therapies are then compared to predict whether the first therapy will have greater deleterious effects in a patient than the second therapy.

In some embodiments, the first therapy comprises one or more therapeutic agents of interest while the second therapy does not comprise the therapeutic agent or therapeutic agents of interest. Accordingly, the methods of the invention may be used to determine whether a first therapy comprising one or more therapeutic agents of interest will have greater therapeutic efficacy or have an increased risk of deleterious effects in comparison with a second therapy that does not comprise the therapeutic agent or therapeutic agents of interest. In alternate embodiments, both the first therapy and the second therapy comprise the same therapeutic agents, but the dosage of one or more of the therapeutic agents in the first therapy differs from the dosage of the same therapeutic agent in the second therapy. Thus, the methods of the invention may also be used to determine whether a first therapy comprising a particular dosage of one or more therapeutic agent or therapeutic agents of interest will have increased therapeutic efficacy or increased risk of deleterious effects in comparison with a second therapy that comprises a different dosage of the therapeutic agent or therapeutic agents of interest. As used herein, a “therapeutic agent” is any compound or agent which is used or contemplated for use in the treatment of a selected condition.

Expression Levels and Expression Profiles

As used herein, an “expression level” or “level of expression” is a value that corresponds to a measurement of the abundance of a gene expression product. Such values may include measurements of RNA levels or protein abundance. Thus, an expression level can be a value that reflects the transcriptional state or the translation state of a gene. The transcriptional state of a sample includes the identities and abundance of the RNA species, especially mRNAs present in the sample. The transcriptional state can be conveniently determined by measuring transcript abundance by any of several existing gene expression technologies. Translational state includes the identities and abundance of the constituent protein species in the sample. As is known to those of skill in the art, the transcriptional state and translational state are related.

In some embodiments, the methods of the present invention comprise providing an expression profile from a sample from a patient. As used herein, an “expression profile” comprises one or more values corresponding to a measurement of the abundance of one or more gene expression products. See, for example, U.S. Pat. Nos. 6,040,138, 5,800,992, 6,020135, 6,344,316, and 6,033,860, which are hereby incorporated by reference in their entireties.

The samples used to determine the expression levels for genes and to generate expression profiles of the present invention can be derived from a variety of sources including, but not limited to, single cells, a collection of cells, tissue, cell culture, bone marrow, blood, or other bodily fluids. The tissue or cell source may include a tissue biopsy sample, a cell sorted population, cell culture, or a single cell. In some embodiments, the samples of the invention are derived from a human patient, while in other embodiments, the samples are derived from a model organism useful for studying a particular disease. Examples of such model organisms include, but are not limited to, mammalian model organisms including rodent model systems and primate model systems.

In selecting a sample, the percentage of the sample that constitutes cells having differential gene expression pre- vs. post therapy (i.e., the cells that are affected by the condition being treated or affected by the selected therapy) should be considered. Samples may comprise at least 20%, at least 30%, at least 40%, at least 50%, at least 55%, at least 60%, at least 70%, at least 75%, at least 80%, at least 85%, at least 90%, or at least 95% cells having expression changes following therapy, with a preference for samples having a higher percentage of such cells.

Where the goal is to find a target for improving activity against a selected condition, samples are preferably taken from cells affected by the selected condition. For example, where the selected condition is a type of solid tumor the sample will preferably be derived from tumor tissue and will comprise tumor cells. Such samples may comprise at least 20%, at least 30%, at least 40%, at least 50%, at least 55%, at least 60%, at least 70%, at least 75%, at least 80%, at least 85%, at least 90%, or at least 95% cells affected by the selected condition with a preference for samples having a higher percentage of such cells. The targets identified based on the differential expression from such samples pre- and post-therapy are used to screen for compounds that synergize or enhance the effect of the selected therapy on expression of the identified target. The identified targets may also be used to screen for compounds that interact with targets downstream of the target of the selected therapy, where such compounds may be useful as a therapeutic agent for the treatment of the condition. Target genes identified from such samples based on a reduction in expression following therapy are used to screen for compounds that will further reduce expression of the target gene and enhance the associated therapeutic effect. Alternatively, target genes identified based on an increase in expression following therapy are used to screen for compounds that can further enhance expression of the target gene.

Where the goal is to find a target to screen for compounds that lessen the deleterious effects caused by the selected therapy, samples are preferably taken from cells that are affected by the deleterious effect. The targets identified based on the differential expression from such samples pre- and post-therapy are used to screen for compounds that inhibit the effect of the selected therapy on expression of the identified target and thereby inhibit the associated deleterious effect. Target genes identified from such samples based on a reduction in expression following therapy are used to screen for compounds that will enhance expression of the target gene and lessen the deleterious effect. Alternatively, target genes identified from such samples based on an increase in expression following therapy are used to screen for compounds that can inhibit expression of the target gene and lessen the side effect.

In some embodiments of the invention, it is preferable but not essential to determine the pre-therapy gene expression level from a sample taken immediately preceding administration of therapy, although any sample taken after the onset of the condition and prior to therapy may be used. When performing the method with a cohort of patients whose differential expression is to be compared, samples should be taken at about the same time relative to therapy administration.

Determination of the post-therapy gene expression levels may be made from a sample taken at any time following treatment with the therapy. Samples will preferably be taken within one to thirty days of therapy administration. The optimum time for taking this sample is contemplated to vary depending on the selected condition, therapy used, and timing of additional confounding therapies. The preferred time may be determined by taking samples at various intervals of time following therapy (and before any additional confounding therapy is administered) and determining which sample provides the largest differential in expression relative to the pre-therapy sample. Accordingly, in some embodiments the sample is taken from the patient within one hour, within two hours, within four hours, within eight hours, within twelve hours, within eighteen hours, within twenty-four hours, within thirty-six hours, within forty-eight hours, within sixty hours, within seventy-two hours, or within ninety-six hours after treatment with the selected therapy. In other embodiments, the sample is taken from the patient within one week, within two weeks, within three weeks, within four weeks, within five weeks, within six weeks, within seven weeks, or within eight weeks after treatment. In still other embodiments, the sample is taken from the patient within two months, within three months, within four months, within six months, within eight months, within ten months, or within a year after treatment.

The expression profiles of the invention comprise one or more values representing the expression level of a gene that is differentially expressed before and after treatment of a selected condition with a selected therapy. By “differentially expressed” it is intended that the expression level of the gene changes significantly after treatment with the selected therapy in comparison with the expression level of the gene before the selected therapy. The expression level may be significantly increased after therapy or significantly decreased after therapy. By a “significant” change in expression level, it is intended a change in expression level that is statistically significant. A statistical test may be used to test whether a change in expression level measured for a gene after treatment is more likely to result from an actual change in the expression of the gene rather than from any variability present in the experimental system.

In an additional aspect of the invention, a patient's response to the subject therapy is also used as a factor in identifying candidate targets. In this aspect, a gene whose pre- vs. post-therapy change in expression is significantly different in patients who did not respond favorably to said therapy (i.e. unresponsive patients, e.g. patients who relapse) compared to patients who did respond favorably to the therapy (i.e. responsive patients) is identified, along with its expression products, as a screening target for drugs which may be used to improve treatment of said selected condition with said selected therapy. Thus, a gene whose expression is increased after therapy in patients who did not respond to therapy and is decreased or unchanged after therapy in responsive patients is identified as a screening target for drugs which can inhibit this increase and lessen the risk of nonresponsiveness to this therapy. Alternatively, a gene whose expression is decreased after therapy in nonresponsive patients and is increased or unchanged after therapy in responsive patients is identified as a screening target for drugs which can prevent this decrease. As yet another example, a gene whose expression is unchanged after therapy in nonresponsive patients and is increased or decreased after therapy in responsive patients is identified as a screening target for drugs which can cause this gene to respond in the same manner observed for responsive patients.

Thus, in some embodiments, the methods of the present invention encompass identifying genes whose expression levels are correlated with a particular treatment outcome or response to treatment with a selected therapy and expression profiles comprising these genes. For example, genes whose levels of expression are associated with a favorable or unfavorable response to a therapy in a patient, or with a deleterious effect of a therapy in a patient may be identified. By a “favorable response” to treatment, it is intended any mitigation or reduction of at least one of symptom associated with the condition to be treated. For example, in the case of cancer, any decrease in the number of cells showing the characteristics of cancer cells would be considered a favorable response to the treatment. By an “unfavorable response” to treatment, it is intended that the treatment does not mitigate or reduce any symptom of the condition. For example, in the case of cancer, an unfavorable response to treatment would include one in which the number of cells showing characteristics of cancer cells did not decrease.

By a gene whose expression level is “correlated with” a particular treatment outcome, it is intended a gene whose expression shows a statistically significant correlation with the treatment outcome. The significance of the correlation between the expression level of a differentially expressed gene and a particular physiologic state such as a favorable or unfavorable response to therapy may be determined by a statistical test of significance. Such methods are known in the art and examples are provided elsewhere herein. Methods for determining the strength of a correlation between the expression level of a differentially-expressed gene and a particular physiologic state are also reviewed in Holloway et al. (2002) Nature Genetics Suppl. 32:481-89, Churchill (2002) Nature Genetics Suppl. 32:490-95, Quackenbush (2002) Nature Genetics Suppl. 32: 496-501; Slonim (2002) Nature Genetics Suppl. 32:502-08; and Chuaqui et al. (2002) Nature Genetics Suppl. 32:509-514; each of which is herein incorporated by reference in its entirety. Such methods may be used to select the genes whose expression levels have the greatest correlation with a particular treatment outcome in order to increase the predictive accuracy of the methods of the invention.

The expression profiles of the invention comprise values representing the absolute or the relative expression level of one or more differentially expressed genes. The expression levels of these genes may be determined by any method known in the art for assessing the expression level of an RNA or protein molecule in a sample. For example, expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. The gene expression monitoring system may also comprise nucleic acid probes in solution.

In one embodiment of the invention, microarrays are used to measure the values to be included in the expression profiles. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, the Examples section. See also, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Determination of the gene expression profile of a sample may be accomplished by any standard means available in the art. One standard way of simultaneously determining the expression profile of a multitude of genes is through the use of arrays. Arrays comprise capture probes for detecting the differentially expressed genes. By “array” is intended a solid support or substrate with peptide or nucleic acid probes attached to said support or substrate. Arrays typically comprise a plurality of different nucleic acid or peptide capture probes that are coupled to a surface of a substrate in different, known locations. These arrays, also described as “microarrays” or colloquially “chips” have been generally described in the art, for example, in U.S. Pat. Nos. 5,143,854, 5,445,934, 5,744,305, 5,677,195, 6,040,193, 5,424,186, 6,329,143, and 6,309,831 and Fodor et al. Science 251:767-77 (1991), each of which is incorporated by reference in its entirety. These arrays may generally be produced using mechanical synthesis methods or light directed synthesis methods that incorporate a combination of photolithographic methods and solid phase synthesis methods.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

The arrays used to practice the methods of the present invention comprise capture probes that can specifically bind a nucleic acid molecule that is differentially expressed in pre-therapy patient samples vs. post-therapy patient samples, or a nucleic acid molecule that is differentially regulated after therapy in patients who relapse after a selected therapy compared to patients who respond favorably to the selected therapy. These arrays can be used to measure the expression levels of nucleic acid molecules to thereby create an expression profile for use in methods of identifying screening targets for drugs that can be used to improve the selected therapy.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample. See, for example, the Examples.

In another embodiment, the values in the expression profile are obtained by measuring the abundance of the protein products of the differentially-expressed genes. The abundance of these protein products can be determined, for example, using antibodies specific for the protein products of the differentially-expressed genes. The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin. The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length protein product from a differentially-expressed gene, or an antigenic peptide fragment of the protein product can be used as an immunogen. Preferred epitopes encompassed by the antigenic peptide are regions of the protein product of the differentially expressed gene that are located on the surface of the protein, e.g., hydrophilic regions, as well as regions with high antigenicity. The antibody can be used to detect the protein product of the differentially expressed gene in order to evaluate the abundance and pattern of expression of the protein. These antibodies can also be used diagnostically to monitor protein levels in tissue as part of a clinical testing procedure, e.g., to, for example, determine the efficacy of a given therapy.

Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, b-galactosidase, or acetylcholinesterase;

examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The present invention encompasses methods in which the expression level or expression profile for a patient are measured before and after treatment. The present invention also provides methods comparing the changes in pre- and post-treatment expression levels for populations of patients. Such populations of patients may comprise two or more patients. Methods are known in the art for comparing two or more data sets to detect similarity between them. To determine whether two or more gene expression levels, fold changes in gene expression or expression profiles show statistically significant similarity, statistical tests may be performed to determine whether any differences between the expression levels, fold changes in gene expression, or expression profile are likely to have been achieved by a random event. Methods for comparing gene expression profiles to determine whether they share statistically significant similarity are known in the art and also reviewed in Holloway et al. (2002) Nature Genetics Suppl. 32:481-89, Churchill (2002) Nature Genetics Suppl. 32:490-95, Quackenbush (2002) Nature Genetics Suppl. 32: 496-501; Slonim (2002) Nature Genetics Suppl. 32:502-08; and Chuaqui et al. (2002) Nature Genetics Suppl. 32:509-514; each of which is herein incorporated by reference in its entirety.

Methods of Identifying Genes and Their Expression Products as Targets in Drug Screening.

The present invention demonstrates that patients affected by the same condition show different expression profiles in response to treatment with different therapeutic regimens. In addition, patients share common pathways of genomic response to the same treatment. Accordingly, the present invention provides methods for identifying one or more genes and their expression products as screening targets for drugs that may be used to treat a selected condition or to improve treatment of a selected condition with a selected therapy. The methods involve measuring gene expression levels of one or more genes in a subject affected by a condition of interest before and after treatment.

In some embodiments, the methods comprise the steps of:

1. determining the expression level of one or more genes in a first sample from a subject affected by the selected condition prior to treatment with the selected therapy;

2. determining the expression level of said one or more genes in a second sample from said subject following said treatment with the selected therapy; and

3. for each of said one or more genes, comparing the expression level measured in step 1 with the expression level measured in step (2).

In the methods, a gene whose expression level is significantly increased or significantly decreased following treatment with the selected therapy is identified, along with its expression products, as a screening target for drugs which may be used to improve treatment of the selected condition with the selected therapy.

In some embodiments of the invention, pre- and post-therapy expression levels are measured in a population of patients. By a “population of patients” is intended one or more patient affected by the same conditions. The number of patients to be included in the population varies according to the selected condition and selected therapy. In some embodiments, it will be sufficient to compare pre-and post-therapy levels in a single patient in order to identify genes whose expression level changes after treatment with the therapy. In other embodiments, a larger population of patients may be used to increase the accuracy for identifying genes that are differentially expressed pre- and post-therapy. Accordingly, the population of patients comprises at least one patient, and may also comprise at least two patients, at least three patients, at least four patients, at least five patients, at least six patients, at least eight patients, at least ten patients, at least fifteen patients, at least twenty-five patients, at least fifty patients, at least one hundred patients, at least two hundred patients, and least three hundred patients, at least five hundred patients, at least one thousand patients, or at least ten thousand patients.

Thus, in some embodiments of the invention, the methods comprise the additional steps of repeating steps 1, 2, and 3 of the method recited above for each subject in a population of subjects affected by the selected condition and comparing the genes whose levels of expression are significantly increased or significantly decreased following treatment with the selected therapy for the subjects in the population of patients affected by the selected condition to thereby identify genes whose levels of expression are correlated with the selected therapy, where a gene whose expression level is correlated with the selected therapy is identified, along with its expression products, as a screening target for drugs which may be used to treat the selected condition or to improve treatment of the selected condition with the selected therapy. Accordingly, in some embodiments, the screening targets identified by the methods are used to identify drugs that can be used in combination with the selected therapy to improve the patient response to selected therapy, while in other embodiments, the screening targets are used to identify drugs that can replace the selected therapy (e.g., drugs that act down stream of the selected therapy) and can be used independently of the selected therapy to treat the condition.

In other embodiments of the invention, the methods comprise the additional steps of determining which subjects responded favorably to the selected therapy and which subjects did not respond favorably to the selected therapy; and comparing the genes showing a change in expression level following treatment with the selected therapy in subjects who responded favorably to the selected therapy and genes showing a change in expression level following treatment with the selected therapy in subjects who did not respond favorably to the selected therapy, to thereby identify genes whose expression level is correlated with a favorable response to the selected therapy. In accordance with the method, a gene whose expression level is correlated with favorable response in a patient to the selected therapy is identified, along with its expression products, as a screening target for drugs that may be used to improve treatment of the selected condition with the selected therapy.

The invention also provides methods for using expression profiles to identify genes and their expression products as screening targets for drugs that may be used to improve treatment of a selected condition with a selected therapy. The methods comprise the steps of:

1. providing a first expression profile comprising values representing the expression levels of one or more genes from a first sample from a subject affected by the selected condition prior to treatment with the selected therapy;

2. providing a second expression profile comprising values representing the expression levels of said one or more genes from a second sample from said subject, wherein said second sample is taken from said patient following treatment with the selected therapy;

3. comparing the values comprised in the first expression profile with those comprised in the second expression profile;

According to the method, a gene whose expression level is significantly increased or significantly decreased following treatment with the therapy is identified, along with its expression products, as a screening target for drugs which may be used to improve treatment of the selected condition with the selected therapy.

The invention provides methods for identifying genes and their expression products as screening targets for inhibitors that may be used to treat a selected condition or to improve treatment of a selected condition with a selected therapy. The methods comprise determining expression levels of one or more genes before and after treatment with a selected therapy for a population of subjects to identify genes whose expression level is significantly increased following therapy, determining which subjects responded favorably to the selected therapy and which subjects did not respond favorably to the selected therapy; and comparing the genes whose expression level is significantly increased following treatment with the selected therapy in subjects who responded favorably to the selected therapy with the genes whose expression level is significantly increased following treatment with the selected therapy in subjects who did not respond favorably to the selected therapy, to thereby identify genes for which a significant increase in expression level following treatment with the selected therapy is correlated with a failure to respond favorably to the selected therapy. A gene whose expression level is correlated with an unfavorable response to the selected therapy is identified, along with its expression products, as a screening target for inhibitors that may be used to improve treatment of the selected condition with the selected therapy.

In other embodiments, the invention provides methods for identifying genes and their expression products as screening targets for mimics or activators that may be used to treat a selected condition or improve treatment of a selected condition with a selected therapy comprising. The methods comprise determining expression levels of one or more genes before and after treatment with a selected therapy for a population of subjects to identify genes whose expression level is decreased following treatment with the therapy, determining which subjects responded favorably to the selected therapy and which subjects did not respond favorably to the selected therapy; and comparing the genes whose expression level is significantly decreased following treatment with the selected therapy in subjects who responded favorably to the selected therapy with the genes whose expression level is significantly decreased following treatment with the selected therapy in subjects who did not respond favorably to the selected therapy, to thereby identify genes for which a significant decrease in expression level following treatment with the selected therapy is correlated with a failure to respond favorably to the selected therapy. A gene whose expression level is correlated with a failure to respond favorably to the selected therapy is identified, along with its expression products, as a screening target for mimics or activators which may be used to treat the selected condition or to improve treatment of the selected condition with the selected therapy.

In other embodiments, the invention provides methods for identifying genes and their expression products as screening targets for modulators that may be used to treat a selected condition or improve treatment of a selected condition with a selected therapy comprising. Such methods comprise determining expression levels of one or more genes before and after treatment with a selected therapy for a population of subjects to identify genes whose expression level is significantly changed after treatment, determining which patients responded favorably to the selected therapy and which subjects did not respond favorably to the selected therapy; and comparing the genes whose expression level is significantly changed following treatment with the selected therapy in subjects who responded favorably to the selected therapy with the genes whose expression level is significantly changed following treatment with the selected therapy in subjects who did not respond favorably to the selected therapy to thereby identify genes for which a significant change in expression level following treatment with the selected therapy is correlated with a failure to respond favorably to the selected therapy. According to the method, a gene whose expression level is significantly changed post-treatment in patients who responded favorably to the selected therapy but whose expression level did not significantly change post-treatment in patients who did not respond favorably to the selected therapy is identified, along with its expression products, as a screening target for modulators which may be used to improve treatment of the selected condition with the selected therapy.

In each method, pre-and post-treatment gene expression levels may be compared by determining the expression levels of one or more genes, or by comparing expression profiles derived from samples taken before and after treatment. The condition for which treatment is provided in the methods may be any condition, including, as non-limiting examples, cancers, genetic disorders, infectious diseases (including viral and bacterial infections), hematological disorders, cardiovascular diseases, dermatological diseases, endocrine diseases and gastrointestinal disorders. The samples from the subjects will typically comprise cells having differential gene expression pre- and post-therapy, for example cells that are affected by the condition being treated or the therapy being used.

Methods of Evaluating Therapies

It is the novel finding of the present invention that administration of a combination therapy comprising multiple therapeutic agents can alter the nature of cellular genomic response when compared with the response to any of the therapeutic agents given alone, and that this cellular genomic response is distinct from the sum of the individual therapeutic agents. Accordingly, the present invention provides methods for predicating the therapeutic efficacy and the likelihood for deleterious effects for therapies based on pre- and post-therapy gene expression levels. By “therapeutic efficacy” it is intended the ability of the therapy to alleviate (e.g., mitigate, decrease, reduce) at least one of the symptom associated with the condition to be treated. By “deleterious effects” of a therapy, it is intended any change in the physiologic state of the patient caused by the therapy that does not contribute to the therapeutic efficacy of the therapy.

In one embodiment, the invention provides a method for predicting whether a first therapy will have increased therapeutic efficacy in a patient in comparison with a second therapy. The method comprises the steps of:

1) determining the expression level of one or more genes in a first sample from a subject affected by the selected condition prior to treatment with the first therapy, wherein increased or decreased expression of said one or more genes after treatment is correlated with a favorable response in a subject to treatment;

2) determining the expression level of said one or more genes in a second sample from the subject of (1) following said treatment with the first therapy;

3) for each of said one or more genes, comparing the expression level measured in step (1) with the expression level measured in step (2) to determine the change in the expression level of said genes following treatment with the selected therapy;

4) repeating steps (1), (2), and (3) for each patient in a population of patients affected by the selected condition; and

5) determining the expression level of said one or more genes in a first sample from a subject affected by the selected condition prior to treatment with the second therapy;

6) determining the expression level of said one or more genes in a second sample from the subject of (5) following said treatment with the second therapy;

7) for each of said one or more genes, comparing the expression level measured in step (5) with the expression level measured in step (6) to determine the change in the expression level of said genes following treatment with the selected therapy;

8) repeating steps (5), (6), and (7) for each patient in a population of patients affected by the selected condition; and

9) for each of said one or more genes, comparing the change in expression level following treatment with the first therapy with the change in expression level following treatment with the second therapy combination therapy to thereby determine whether the expression levels of the one or more genes show a greater increase in expression levels following treatment with the first therapy than following treatment with the second therapy. For genes for which increased expression following treatment is correlated with a favorable response in a subject to treatment, a greater increase in expression levels for one or more of the genes following treatment with the first therapy in comparison with the expression level for the one or more genes following treatment with the second therapy results in a prediction that the first therapy will have increased therapeutic efficacy in a patient in comparison with the second. For genes for which decreased expression following treatment is correlated with a favorable response in a subject to treatment, a greater decrease in expression levels for one or more of the genes following treatment with the first therapy in comparison with the expression level for the one or more genes following treatment with the second therapy results in a prediction that the first therapy will have increased therapeutic efficacy in a patient in comparison with the second therapy.

The genes whose expression levels are measured in the method may be any genes showing differential expression following treatment of the condition with any therapy. In some embodiments, a change in the expression of the genes following treatment is correlated with a favorable response following treatment with the first therapy. In other embodiments, a change in the expression of the genes following treatment is correlated with a favorable response following treatment with the second therapy. In still other embodiments, a change in the expression of the genes following treatment is correlated with a favorable response to treatment of in response to a therapy other than the first therapy or second therapy to be tested.

In another embodiment, the invention provides a method for predicting whether a first therapy will have increased deleterious effects in a patient in comparison with a second therapy. The method comprises the steps of:

1) determining the expression level of one or more genes in a first sample from a subject affected by the selected condition prior to treatment with the first therapy, wherein increased or decreased expression of said one or more genes after treatment is correlated with deleterious effects in a subject to in response to treatment;

2) determining the expression level of said one or more genes in a second sample from the subject of (1) following said treatment with the first therapy;

3) for each of said one or more genes, comparing the expression level measured in step (1) with the expression level measured in step (2) to determine the change in the expression level of said genes following treatment with the selected therapy;

4) repeating steps (1), (2), and (3) for each subject in a population of subjects affected by the selected condition; and

5) determining the expression level of said one or more genes in a first sample from a subject affected by the selected condition prior to treatment with the second therapy;

6) determining the expression level of said one or more genes in a second sample from the subject of (5) following said treatment with the second therapy;

7) for each of said one or more genes, comparing the expression level measured in step (5) with the expression level measured in step (6) to determine the change in the expression level of said genes following treatment with the selected therapy;

8) repeating steps (5), (6), and (7) for each subject in a population of subjects affected by the selected condition; and

9) for each of said one or more genes, comparing the change in expression level following treatment with the first therapy with the change in expression level following treatment with the second therapy to thereby determine whether the expression levels of said one or more genes show a greater increase in expression levels following treatment with said first therapy than following treatment with the second therapy. For genes for which increased expression following treatment is correlated with a deleterious effects in a patient to treatment, a greater increase in expression levels for one or more of the genes following treatment with the first therapy in comparison with the expression level for the one or more genes following treatment with the second therapy results in a prediction that the first will have increased deleterious effects in a patient in comparison with the second therapy. For genes for which increased expression following treatment is correlated with deleterious effects in a patient to treatment, a greater decrease in expression levels for one or more of the genes following treatment with the first therapy in comparison with the expression level for the one or more genes following treatment with the second therapy results in a prediction that the first therapy will have increased deleterious effects in a patient in comparison with the second therapy

The genes whose expression levels are measured in the method may be any genes showing differential expression following treatment of the condition with the any therapy. In some embodiments, a change in the expression of the genes following treatment is correlated with deleterious effects following treatment with the first therapy. In other embodiments, a change in the expression of the genes following treatment is correlated with deleterious effects following treatment with the second therapy. In still other embodiments, a change in the expression of the genes following treatment is correlated with deleterious effects following treatment with a therapy other than the first therapy or second therapy to be tested.

In some embodiments of the methods of the invention, the genes for which increased or decreased expression after therapy is correlated with a favorable response in a patient to treatment with said a combination therapy are identified by a method comprising:

1) determining the expression level of one or more genes in a first sample from a subject affected by from the selected condition prior to treatment with a first therapy;

2) determining the expression level of said one or more genes in a second sample from said subject following said treatment with said first therapy;

3) for each of said one or more genes, comparing the expression level measured in step (1) with the expression level measured in step (2) to identify genes whose expression level changed significantly following treatment with said first therapy;

4) repeating steps (1)-(3) for each patient in a population of subjects affected by the selected condition;

5) determining which subjects responded favorably to said first therapy and which subjects did not respond favorably to said first therapy; and

6) comparing the genes whose expression level increased significantly or decreased significantly following treatment with said first therapy in subjects who responded favorably to said first therapy with the genes whose expression level did not change significantly following treatment with said first therapy in subjects who did not respond favorably to the said first therapy, to thereby identify genes for which an increase or decrease in expression following treatment with said first therapy is correlated with a favorable response in a subject to said first therapy.

In each method, pre-and post-treatment gene expression levels may be compared by determining the expression levels of one or more genes, or by comparing expression profiles derived from patient samples before and after treatment. The condition for which treatment is provided in the methods may be any condition, including, as non-limiting examples, cancers, genetic disorders, infectious diseases (including viral and bacterial infections), hematological disorders, cardiovascular diseases, dermatological diseases, endocrine diseases and gastrointestinal disorders. The samples from the subjects will typically comprise cells having differential gene expression pre- and post-therapy, for example cells that are affected by the condition being treated or the sample being used.

Methods of Screening for Drugs that Modulate Therapeutic Targets

The differentially expressed genes and their expression products identified as targets in accordance with the invention may be used in conventional biochemical assays or in cell-based screening assays. Johnston, P. A. and Johnston, P. A., “Cellular Platforms for HTS: three case studies”, Drug Discovery Today 7(6): 353-363 (March 2002); Drews, J., “Drug discovery: a historical perspective”, Science 287: 1960-1965 (2000); Valler, M. J. and Green, D., “Diversity screening versus focused screening in drug discovery”, Drug Discovery Today 5(7): 286-293 (2000); Grepin, C. and Pernelle, C., “High-throughput screening”, Drug Discovery Today 5(5): 212-214 (2000); “Recent patents in high-throughput screening”, Nat. Biotechnol. 18(7): 797 (2000); White, R. E., “High-throughput screening in drug metabolism and pharmacokinetic support of drug discovery”, Ann. Rev. Pharmacol. Toxicol. 40: 133-157 (2000); Broach, J. R. and Thorner, J., “High-throughput screening for drug discovery”, Nature 384 (Suppl): 14-16 (1996), Silverman, L. et al., “New assay technologies for high-throughput screening”, Curr. Opin. Chem. Biol. 2:397-403 (1998). Such biochemical assays are based on the activity of the expression product and include standard kinase assays, phosphatase assays, binding assays, assays for apoptosis, hydroxylation, oxidation, conjugation and other enzyme reactions, and assays for protein-protein or protein-DNA or RNA interactions. Cell-based screening assays utilize recombinant host cells expressing the differentially expressed gene product. The recombinant host cells are screened to identify compounds that can activate the product of the differentially expressed gene or increase expression of the gene (i.e. agonists), or inactivate the product of the differentially expressed gene or decrease expression of the gene (i.e. antagonists).

Alternatively, a chimeric gene comprising the coding sequence for a reporter protein, such as green fluorescence protein or luciferase, placed under the regulatory of the promoter of a differentially expressed gene can be made. Such a chimeric gene can be used in a cell-based assay to screen for compounds that enhance or inhibit expression of the reporter gene through regulation of the promoter of the differentially expressed gene. Dhundale, A. and Goddard, C., “Reporter assays in the high throughput screening laboratory: a rapid and robust first look”, J. Biomol. Screening 1:115-118 (1996); Goetz, A. S. et al., “Development of a facile method for high throughput screening with reporter gene assays”, J. Biomol. Screening 5: 377-384 (2000).

Candidate compounds which may be screened for activity against targets identified by practice of the present invention include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries; 5) zinc analogs; 6) leukotriene A₄ and derivatives; 7) classical aminopeptidase inhibitors and derivatives of such inhibitors, such as bestatin and arphamenine A and B and derivatives; 8) and artificial peptide substrates and other substrates, such as those disclosed herein above and derivatives thereof.

The compounds used for screening against targets identified in accordance with the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,223,409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. U.S.A. 97:6378-6382); (Felici (1991) J. Mol. Biol. 222:301-310).

Modulators of the activity of a product of a differentially expressed gene identified according to the drug screening assays provided above can be used to improve treatment of a selected condition. These methods of treatment include the steps of administering the modulators of the activity of a product of a differentially-expressed gene in a pharmaceutical composition as described herein, in combination with the selected therapy, to a subject in need of such treatment.

### EXAMPLES

The following examples are offered by way of illustration and not by way of limitation.

### Example 1

### Treatment-Specific Changes in Gene Expression in Primary Leukemia Cells, In Vivo, During Initial Therapy for Acute Lymphoblastic Leukemia (ALL)

### SUMMARY

To elucidate genomic determinants of leukemia response to chemotherapy, oligonucleotide microarrays (Affymetrix® HG-U95A GeneChip) were used to analyze expression of approximately 9,600 human genes in bone marrow leukemic blasts obtained from children with ALL, at diagnosis and one day post-treatment with mercaptopurine (1 gm/² IV) or methotrexate (MTX) given alone (1 gm/m² IV), or mercaptopurine (6-MP) in combination with either low-dose MTX [180 mg/m² orally] or high-dose MTX [1.0 mg/m² IV]). A stratified (immunophenotype, DNA ploidy) randomization was used to assign treatment, and the fold-change in gene expression (post-treatment to diagnosis) was computed for 60 patients. Using linear discriminate analysis with variance (LDAV) genes that most discriminated among treatments were selected based on expression changes (fold-change from diagnosis to post-treatment) or based on post-treatment expression levels alone. There were distinct expression profiles that discriminated among all treatments, using either the fold-change or the post-treatment expression patterns, although the change in gene expression discriminated significantly better among treatments. Leave-one-out cross-validation using support-vector-machine (SVM), based on the 120 most discriminating genes, correctly classified 60 out of 60 patients (100%) based on fold-change versus 58 out of 60 (96.7%) using only post-treatment expression profiles. The smallest number of genes for discrimination among treatments was 120 using fold-change, which included genes involved in cellular processes such as apoptosis, cell cycle control and stress response. Together, these in vivo data reveal unique, treatment-specific changes in gene expression in primary leukemia cells, establishing that changes in expression differ according to the specific medication, dosage and combination given. These findings provide new insights to cancer cell responses to chemotherapy and can be used to illuminate mechanisms of leukemia resistance and identify novel targets to augment existing treatment modalities.

### Methods

Primary leukemia cells. This study included 60 patients with ALL enrolled on St. Jude Children's Research Hospital Total Therapy Studies XIIIB and XV. Bone marrow samples were obtained at diagnosis (pre-treatment) and one day post-treatment with mercaptopurine (6-MP) or methotrexate (MTX) given alone, or mercaptopurine in combination with either low-dose MTX (LDMTX/6-MP) or high-dose MTX (HDMTX/6-MP). A stratified (immunophenotype, DNA ploidy) randomization was used to assign treatment. Total RNA was extracted from cryopreserved mononuclear cell suspensions with TriReagent (MRC, Cincinnati, Ohio).

Mircoarray analysis. High quality RNA was hybridized to Affymetrix HG-U95A GeneChipe (12,600 probe sets, ˜9,600 human genes) according to the manufacturers protocol (Affymetrix, Santa Clara, Calif.). Scaled gene expression values for pre-treatment, post-treatment and fold-change (post-treatment vs. pre-treatment ratio) were calculated using Affymetrix Microarray Suite® (MAS) 5.0.

Gene expression data analysis. Analysis was done on fold-change and on post-treatment expression. The data were log-transformed and probe sets were filtered out if “absent” in all 120 arrays or if “no change” in all 60 fold-change ratios. Principal component analysis (PCA) and 2D-hierarchical clustering was performed using GeneMath 1.5 (AppliedMaths, Belgium). We applied supervised methods to find the most discriminating genes, including Linear Discriminant Analysis with Variance (LDAV) (GeneMaths) and ANOVA. Probe sets were ranked according to their discriminating power. To establish that these genes could classify treatments and to find significant genes, leave-one-out cross-validation was performed by support vector machine (SVM) with the top ranked probe sets. To demonstrate the above selected genes were not obtained by chance, a permutation test was performed in which each patient was assigned randomly to one of the four treatments groups, and the same procedure was followed to select genes and perform cross-validation. The p-value is defined as the probability of observing a misclassification rate less or equal to that in the experimental data. 250 permutations were performed. To distinguish one treatment from the other treatments, distinction calculation (Spotfire 6.3, Somerville, Mass.) was performed for each probe set. Permutations (n=1000) were performed to obtain the p-values. Among probe sets with p-values <0.01, those with the largest distinction values were selected.

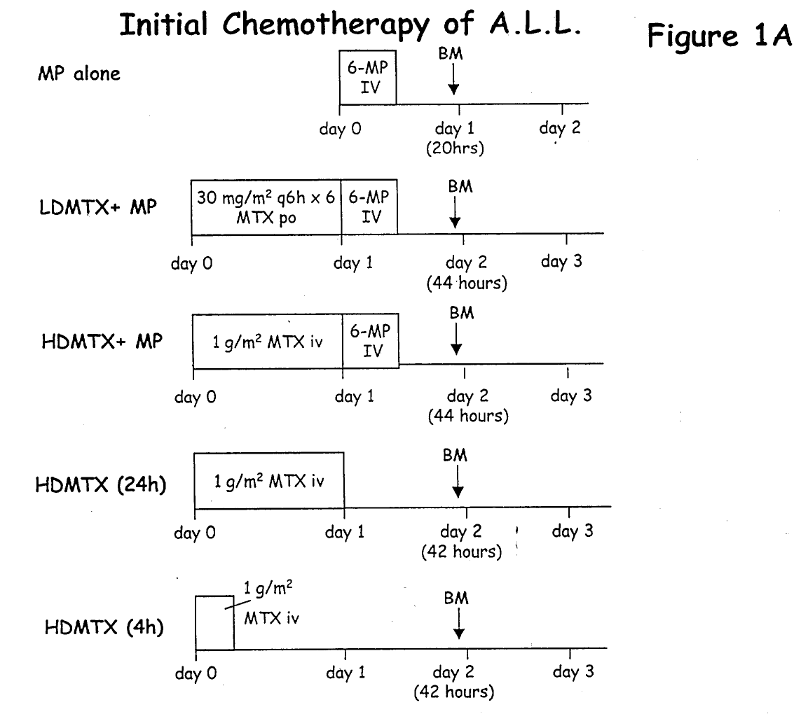

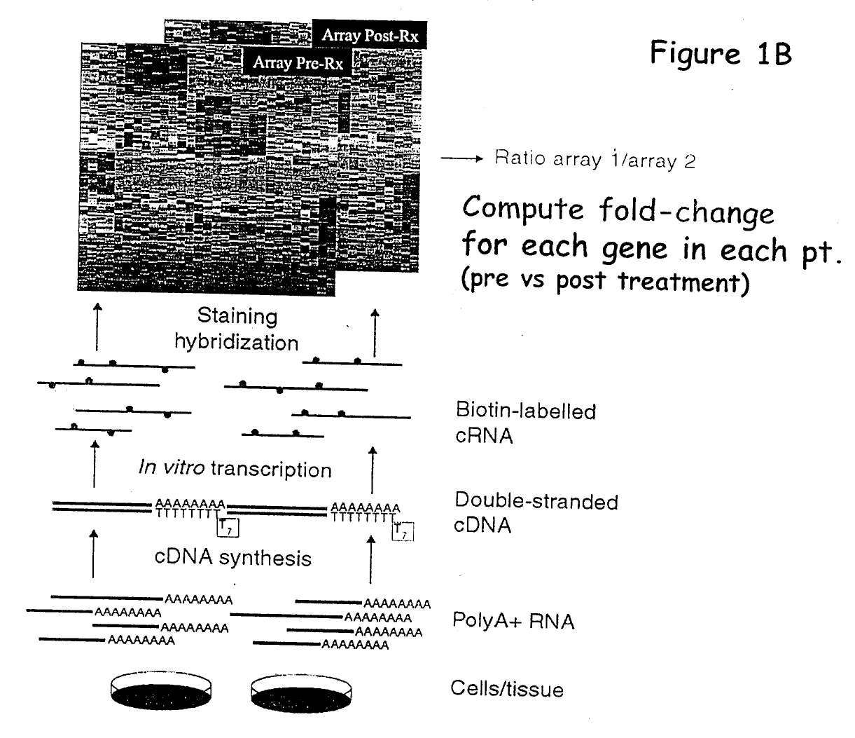

Treatment Regimen and bone marrow sampling time. Bone marrow samples were obtained at diagnosis (pre-treatment) and one day post-treatment with mercaptopurine (6-MP) or methotrexate (MTX) given alone, or mercaptopurine in combination with either low-dose MTX (LDMTX/6-MP) or high-dose MTX (HDMTX/6-MP). After total RNA extraction, samples were processed according to Affymetrix protocol. Fold-change as well as expression values for each gene in each patient were computed. A schematic of this process is shown in FIG. 1A and FIG. 1B.

Patient characteristics. A total of 60 patients were analyzed. No difference was found in terms of gene expression in this study between HDMTX (infusion for 24 h) treatment and HDMTX (infusion for 4 h) treatment. Therefore data from these patients was pooled together.

Unsupervised clustering of 60 ALL samples and PCA using all genes. About 8222 genes (fold-change) and 8002 (post-treatment only) were used for hierarchical clustering. Post-treatment samples clustered by lineage, ploidy and molecular subtypes.

Leave-one-out cross-validation results. SVMs were constructed using top ranked genes. Leave-one-out cross-validation showed that classification error rate decreased as the number of genes used to make the classification increased. Using the 120 genes showing the greatest fold-change in gene expression, all patients were correctly assigned to their corresponding treatment group by this analysis. Selected top 160 genes for post-treatment only, correctly assigned 58 out of 60, the latter indicating that in some cases the changes in gene expression is more informative than just the post-treatment expression profile.

2D-Hierarchical clustering. Using the 120 most discriminating genes based on fold-change observed between pre-treatment and post-treatment expression, a 2-dimensional hierarchical cluster of genes whose change in expression was associated with a particular treatment was created. This exercise identified genes whose change in expression (either up or down) was characteristic of a particular treatment and could be used to determine which treatment had been administered to a particular patient.

Clustering of 60 ALL samples with most discriminating genes only. Three dimensional hierarchical clustering was performed using expression data from A) 120 genes (fold-change) and 160 genes (post-treatment only). Both analyses resulted in clustering of patients according to the treatment they were given, with only one sample being misclassified by this process. Differences between the four treatment groups was more evident from the comparison of fold-change in pre and post treatment gene expression than for post-treatment gene expression alone.

Distinction calculation results. To distinguish one treatment from the other treatments, distinction calculation values were computed. The ten genes with the highest distinction values for both directions (five up-regulated and five down-regulated) for each treatment are shown in the table below. These genes and their expression products represent screening targets that may be used to synergize or enhance the effects of the therapy they are associated with.

TABLE 1
Top 40 Discriminating Genes

| Treatment | GenBank | Name |
|---|---|---|
| HDMTX | W72424 | S100 calcium binding protein A9 |
| | AF004230 | Leukocyte immunoglobulin-like receptor |
| | AL036554 | Defensin, alpha 3, neutrophil-specific |
| | AI126134 | S100 calcium binding protein A8 |
| | AA151971 | CDNA clone = IMAGE-588365 |
| | AB007939 | KIAA0470 gene product |
| | AB024327 | unr-interacting protein |
| | L42542 | ralA binding protein 1 |
| | D64109 | Transducer of ERBB2, 2 |
| | X89750 | TGFB-induced factor |
| HDMTX/ | D88532 | Phosphoinositide-3-kinase |
| 6-MP | L20826 | Plastin 1 (I isoform) |
| | AF003001 | Telomeric repeat binding factor |
| | U93867 | Polymerase (RNA) III (DNA directed) (62kD) |
| | U46116 | Protein tyrosine phosphatase, receptor type. |
| | | G |
| | U66469 | cell growth regulatory with ring finger domain |
| | U51698 | Apoptosis antagonizing transcription factor |
| | Z99716 | Septin 3 |
| | AI701164 | Ubiquitin-conjugating enzyme E2G 1 |
| | AJ006068 | DTDP-D-glucose 4,6-dehydratase |
| LDMTX/ | Y15801 | protein kinase, Y-linked |
| 6-MP | AB014582 | KIAA0682 gene product |
| | L13689 | murine leukemia viral (bmi-1) oncogene |
| | U65416 | MHC class I polypeptide-related sequence B |
| | M77698 | YY1 transcription factor |
| | X66358 | cyclin-dependent kinase-like 1 |
| | X12451 | cathepsin L |
| | W28760 | cDNA/gb = W28760 |
| | AF054177 | chromodomain helicase DNA binding protein |
| | M34379 | elastase 2, neutrophil |
| 6MP | L36720 | bystin-like |
| | AF051941 | nucleoside diphosphate kinase type 6 |
| | X59303 | valyl-tRNA synthetase 2 |
| | AA149307 | hypothetical protein FLJ21174 |
| | L27071 | TXK tyrosine kinase |
| | AA005018 | CGI-49 protein |
| | X54486 | serine (or cysteine) proteinase inhibitor |
| | M28393 | perforin 1 (pore forming protein) |
| | U34683 | glutathione synthetase |
| | U72066 | retinoblastoma binding protein 8 |

HDMTX = Methotrexate administered intravenously at a high dose level of 1 gm/m².
LDMTX = Methotrexate administered intravenously at a low dose level of 180 mg/m².
6-MP = Mercaptopurine administered intravenously at a dose level of 1 gm/m².

Conclusion. Together, these in vivo data reveal unique, treatment-specific changes in gene expression in primary leukemia cells, establishing that changes in expression differ according to the specific medication, dosage and combination given. These findings provide new insights to cancer cell responses to chemotherapy, illuminate mechanisms of leukemia resistance and identify novel targets to augment existing treatment modalities.

### Example 2

### Treatment-Specific Changes in Gene Expression in Primary Leukemia Cells, In Vivo, During Initial Therapy for Acute Lymphoblastic Leukemia (ALL) Associated With Relapse

Gene Expression data from Example 1 was further analyzed according to which patients responded favorably to therapy and which patients suffered from a relapse following therapy. Based on this analyses, genes were identified whose expression was down regulated after therapy administration in patients which subsequently suffered a relapse relative to patients which responded favorably to therapy. These genes are identified in Table 2A below. In accordance with the teachings of the present invention, these genes are identified as targets to screen for drugs that can increase their expression or increase the activity of their expression products. Such drugs could be used to improve the subject ALL therapy.

TABLE 2A
Genes down-regulated in relapse patients

| GenBank | |
|---|---|
| Accession# | Gene Name |
| AF081287 | CTD (carboxy-terminal domain, RNA polymerase II, polypeptide A) phos. |
| U33203 | Mdm2, transformed 3T3 cell double minute 2, p53 binding protein |
| (mouse) | |
| D28532 | solute carrier family 17 (sodium phosphate), member 1 |
| AF052182 | DHHC1 protein |
| M35878 | insulin-like growth factor binding protein 3 |
| AF023466 | glycine-N-acyltransferase |
| W72239 | |
| W28255 | gamma tubulin ring complex protein (76p gene) |
| U44755 | small nuclear RNA activating complex, polypeptide 2,45kD |
| M14502 | arginase, liver |
| D87436 | lipin 2 |
| S80267 | spleen tyrosine kinase |
| M15169 | adrenergic, beta-2, receptor, surface |
| X66397 | translocated promoter region (to activated MET oncogene) |
| AB012293 | lymphocyte antigen 6 complex, locus H |
| W37606 | HCF-binding transcription factor Zhangfei |
| AF056490 | phosphodiesterase 8A |
| AF070617 | |
| D30655 | eukaryotic translation initiation factor 4A, isoform 2 |
| AJ001019 | ring finger protein 3 |
| X83300 | SMA4 |
| AJ007292 | ephrin-A2 |
| D87012 | topoisomerase (DNA) III beta |
| X52151 | arylsulfatase A |
| S66427 | retinoblastoma binding protein 1 |
| D83407 | Down syndrome critical region gene 1-like 1 |
| M90360 | A kinase (PRKA) anchor protein 13 |
| AL050372 | |
| X95152 | breast cancer 2, early onset |
| Y13492 | smoothelin |
| AA151922 | APG12 autophagy 12-like (S.cerevisiae) |
| AI560890 | |
| U29656 | non-metastatic cells 3, protein expressed in |
| AF026029 | poly(A) binding protein, nuclear 1 |
| U69883 | potassium intermediate/small conductance calcium-activated channel.su. |
| AF006513 | chromodomain helicase DNA binding protein 1 |
| AB029032 | KIAA1109 protein |
| U12779 | mitogen-activated protein kinase-activated protein kinase 2 |
| N29665 | KIAA0618 gene product |
| Z29630 | spleen tyrosine kinase |
| W22296 | protein kinase C binding protein 1 |
| U34994 | protein kinase, DNA-activated, catalytic polypeptide |
| AB003791 | carbohydrate (keratan sulfate Gal-6) sulfotransferase 1 |
| AF094521 | Cdc42 effector protein 3 |
| AB026190 | Kelch motif containing protein |
| AF000430 | dynamin 1-like |
| L12723 | heat shock 70kD protein 4 |
| X60592 | tumor necrosis factor receptor superfamily, member 5 |
| AL049787 | hypothetical gene CG018 |
| AF052169 | |

This analysis also revealed genes whose expression was up-regulated after therapy administration in patients which subsequently suffered a relapse relative to patients which responded favorably to therapy. These genes are identified in Table 2B below. In accordance with the teachings of the present invention, these genes are identified as targets to screen for drugs which can decrease their expression or decrease the activity of their expression products. Such drugs could be used to improve the subject ALL therapy.

TABLE 2B
Genes up-regulated in relapse patients

| GenBank | |
|---|---|
| Accession # | Gene Name |
| M34276 | |
| AL050162 | testis derived transcript (3 LIM domains) |
| M31516 | decay accelerating factor for complement (CD55, Cromer blood group |
| sys. | |
| AI004207 | hypothetical protein FLJ00002 |
| AF108145 | MYLE protein |
| AF070554 | |
| X78710 | metal-regulatory transcription factor 1 |
| W25984 | Hypothetical protein TCBAP0758 |
| AL050064 | hypothetical protein FLJ11220 |
| U67615 | Chediak-Higashi syndrome 1 |
| AB007864 | KIAA0404 protein |
| X00734 | tubulin, beta, 5 |
| AJ222801 | sphingomyelin phosphodiesterase 2, neutral membrane (neutral sphingo. |
| AF038179 | hypothetical protein FLJ11191 |
| U77664 | ribonuclease P (38kD) |
| X94630 | CD97 antigen |
| U20982 | insulin-like growth factor binding protein 4 |
| AI535828 | jumping translocation breakpoint |
| U40992 | DnaJ (Hsp40) homolog, subfamily B, member 4 |
| Y15908 | diaphanous homolog 2 (Drosophila) |
| AF023456 | protein phosphatase, EF hand calcium-binding domain 2 |
| D50915 | KIAA0125 gene product |
| AF032862 | hyaluronan-mediated motility receptor (RHAMM) |
| AJ243274 | Kruppel-like factor 12 |
| L13698 | growth arrest-specific 1 |
| L40401 | peroxisomal long-chain acyl-coA thioesterase |
| AL049929 | ATPase, H+ transporting, lysosomal (vacuolar proton pump) |
| | membranes. |
| U35146 | cyclin-dependent kinase-like 2 (CDC2-related kinase) |
| AL046940 | |
| U43842 | bone morphogenetic protein 4 |
| AF070524 | |
| AJ010841 | thioredoxin-like 2 |
| D45132 | PR domain containing 2, with ZNF domain |
| AB028995 | KIAA1072 protein |
| D14889 | RAB33A, member RAS oncogene family |
| M16942 | major histocompatibility complex, class II, DR beta 4 |
| W27944 | Wnt inhibitory factor-1 |
| M18728 | carcinoembryonic antigen-related cell adhesion molecule 6 (non-specific. |
| X82209 | meningioma (disrupted in balanced translocation)1 |
| AF023462 | phytanoyl-CoA hydroxylase (Refsum disease) |
| U11821 | tumor necrosis factor (ligand) superfamily, member 6 |
| Y00816 | complement component (3b/4b) receptor 1 including Knops blood group. |
| X53004 | glycophorin E |

### Example 3

### Changes in Gene Expression After Combination Therapy Were Not the Composite of Each Agent Given Alone.

To determine whether changes in gene expression differed when HDMTX or MP were given alone versus in combination, we compared genes that changed expression (by >50%) in over 70% of patients after single agent and combination treatment. In over 70% of patients treated with HDMTX, MP, or HDMTX+MP, 97, 197 and 173 genes changed expression by at least 50%. However, only seven (11.9%) of 59 genes that were down-regulated after HDMTX alone were also down-regulated when HDMTX was given with MP, and only eight (21.1%) of 38 genes that were up-regulated after HDMTX alone also increased after HDMTX+MP. Similarly, only 18 (11.4%) of 158 genes that increased after MP alone also increased after MP+HDMTX, and only seven (17.5%) of 40 genes that were down-regulated after MP alone were also down-regulated after MP+HDMTX. Overall, only 40 of 295 genes (13.6%) that changed after HDMTX alone or MP alone (sum of the two groups) also changed after the combination of HDMTX+MP (Tables 3A and 3B). Among the 295 genes that changed significantly after 6 MP alone or HDMTX alone, the overall magnitude of change in-expression was significantly less after the combination of MP+HDMTX (P<0.001, paired t-test).

TABLE 3A
Genes That Concordantly Change after treatment with HDMTX
Alone and After Treatment with HDMTX-MP*

| | | | Median | Median |
|---|---|---|---|---|
| | | | FC | FC |
| | | | HDMT | HDMTX/ |
| Probe set ID | Identifier | Gene name | X** | MP** |
| 36161_at | M34175 | adaptor-related protein complex 2, beta 1 subunit | 5.7 | 3.6 |
| 37277_at | U80017 | baculoviral IAP repeat-containing 1 | 2.6 | 2.4 |
| 38819_at | U33635 | PTK7 protein tyrosine kinase 7 | 2.5 | 2.4 |
| 36651_at | X15525 | acid phosphatase 2, lysosomal | 2.5 | 1.9 |
| 40123_at | D87435 | golgi-specific brefeldin A resistance factor 1 | 2.2 | 2.0 |
| 34279_at | AL050141 | hypothetical protein FLJ20719 | 2.0 | 2.5 |
| 32125_at | AA928996 | Tho2 | 1.9 | 3.0 |
| 38464_at | X87237 | glucosidase I | 1.9 | 3.2 |
| 36432_at | AL079298 | methylcrotonoyl-Coenzyme A carboxylase 2 (beta) | −1.8 | −2.0 |
| 35074_at | AF004715 | jerky homolog-like (mouse) | −1.9 | −2.5 |
| 36246_at | Z35309 | adenylate cyclase 8 (brain) | −2.2 | −2.8 |
| 32413_at | M13934 | | −2.8 | −2.4 |
| 32583_at | J04111 | v-jun sarcoma virus 17 oncogene homolog (avian) | −3.5 | −2.3 |
| 725_i_at | J03071 | | −4.0 | −5.5 |
| 1915_s_at | V01512 | | −5.7 | −2.4 |

*Only 21.1% of genes (8/38) that were upregulated after HDMTX alone were also upregulated when HDMTX was given with MP, and only 11.9% of genes that were down regulated after HDMTX alone were also down-regulated after HDMTX when given with MP.
**Negative numbers indicate a decrease and positive an increase in expression

TABLE 3B
Genes that Concordantly Change After MP Alone And After
HDMTX + MP*

| | | | | Median |
|---|---|---|---|---|
| | | | Median | FC |
| | | | FC | HDMTX/ |
| Probe set ID | Identifier | Gene Name | MP** | MP** |
| 36161_at | M34175 | adaptor-related protein complex 2, beta 1 subunit | 4.0 | 3.6 |
| 32125_at | AA928996 | Tho2 | 2.5 | 3.0 |
| 35436_at | L06147 | golgi autoantigen, golgin subfamily a, 2 | 2.1 | 2.5 |
| 34836_at | U18420 | RAB5C, member RAS oncogene family | 2.0 | 1.9 |
| 36822_at | U51334 | TAF15 RNA polymerase II, TATA box binding protein | 2.0 | 1.9 |
| 37277_at | U80017 | baculoviral IAP repeat-containing 1 | 2.0 | 2.4 |
| 38915_at | AB011135 | KIAA0563 gene product | 2.0 | 1.9 |
| 31652_at | AB023217 | KIAA1000 protein | −1.9 | −2.2 |
| 41117_s_at | AB016243 | solute carrier family 9 (sodium/hydrogen exchanger) | −2.0 | −1.7 |
| 38146_at | AB011107 | zinc finger protein 387 | −2.1 | −3.5 |
| 940_g_at | D12625 | neurofibromin 1 | −2.1 | −1.7 |
| 31785_f_at | U92817 | unnamed HERV-H protein | −2.3 | −2.5 |
| 34702_f_at | M27826 | chorionic somatomammotropin hormone 2 | −2.3 | −2.9 |
| 41303_r_at | AI378632 | Homo sapiens mRNA; cDNA DKFZp564P233 | −2.5 | −3.6 |
| 450_g_at | U66469 | cell growth regulatory with ring finger domain | −2.5 | −1.9 |
| 40590_at | AA166687 | cell division cycle 27 | −2.8 | −7.7 |
| 31529_at | X99141 | keratin, hair, basic, 3 | −3.0 | −2.1 |
| 39407_at | M22488 | bone morphogenetic protein 1 | −3.0 | −4.3 |
| 33047_at | AI971169 | ESTs, Highly similar to BCL2-like 11 | −3.2 | −2.1 |
| 34704_r_at | AA151971 | chorionic somatomammotropin hormone 2 | −3.5 | −3.0 |
| 40387_at | U80811 | endothelial differentiation,G-protein-coupled receptor | −3.5 | −3.7 |
| 32583_at | J04111 | v-jun sarcoma virus 17 oncogene homolog (avian) | −3.7 | −2.3 |
| 39586_at | AF097935 | desmoglein 1 | −4.0 | −3.0 |
| 1915_s_at | V01512 | | −4.9 | −2.4 |
| 725_i_at | J03071 | | −9.2 | −5.5 |

*Only 17.5% of genes (7/40) that were up-regulated after MP alone were also up-regulated when MP was given with HDMTX and only 11.4% of genes (18/157) that were down-regulated after MP alone were also down-regulated after MP when given with HDMTX.
**Negative numbers indicate a decrease and positive an increase in expression.

### Example 3

### Human Leukemia Cell Lines Differ From Primary Leukemia Cells in Response to Therapy

When the treatments with HDMTX alone (12 nM×24 hr plus 18 hr drug-free media) or MP alone (10 μM×24 hr) were recapitulated with two human ALL cell lines in vitro (i.e., B-lineage Nalm6 [N.MTX] and T-lineage CEM [C.MTX}), very little overlap was found in the genes that changed by >50% after treatment in the cell lines compared to the primary leukemia cells in patients. Specifically, only seven out of the 97 genes (7.2%) that changed by >50% in at least 70% of patients after HDMTX also changed in the cell lines. Similarly, only 27 of the 197 genes (13.7%) changed in a consistent manner after MP treatment of cell lines and primary cells in vivo (see Supplemental Table 4A for list of genes).

TABLE 4A
Genes that Concordantly Change After HDMTX in Cell lines and in
Patients, in vivo *

| (1) | | | Median | Median | Median |
|---|---|---|---|---|---|
| Probe set | | | FC | FC | FC |
| ID | Identifier | Gene name | N.MTX** | C.MTX** | HDMTX** |
| 32264_at | L23134 | granzyme M (lymphocyte met-ase 1) | 1.9 | 1.6 | 2.9 |
| 36591_at | X06956 | tubulin, alpha 1 (testis specific) | 1.9 | 1.2 | 1.9 |
| 33143_s_at | U81800 | solute carrier family 16 (monocarboxylic | 1.5 | 1.7 | 3.6 |
| | | acid transporters), | | | |
| 2067_f_at | L22475 | BCL2-associated X protein | 7.0 | 1.0 | 3.7 |
| 2001_g_at | U26455 | ataxia telangiectasia mutated | 2.1 | 1.6 | 2.5 |
| 35692_at | AL0802 | Ras-induced senescence 1 | −2.6 | −1.0 | −2.5 |
| | 35 | | | | |
| 1916_s_at | V01512 | v-fos FBJ murine osteosarcoma viral | −1.4 | −1.9 | −11.3 |
| | | oncogene homolog | | | |

*Only seven out of the 97 genes (7.2%) that changed by >50% in at least 70% of patients after HDMTX also changed on average by >50% in the cell lines.
**Negative numbers indicate a decrease and positive an increase in expression.

When the treatment with MP alone (10 μM×24 hr.) were recapitulated with two human ALL cell lines in vitro (i.e., B-lineage Nalm6 [N.MP] and T-lineage CEM [C.MP}), very little overlap was found in the genes that changed after treatment in the cell lines compared to the primary leukemia cells in patients. The genes that concordantly change after HDMTX in cell lines and in patients are listed in Table 4B.

TABLE 4B
Genes that Concordantly Change after HDMTX in cell lines and in
patients*

| | | | Median | Median | Median |
|---|---|---|---|---|---|
| Probe set | | | FC | FC | FC |
| ID | Identifier | Gene name | N.MP** | C.MP** | MPII** |
| 37881_at | AF1009 | growth differentiation factor 11 | 2.6 | 3.7 | 2.1 |
| | 07 | | | | |
| 38547_at | Y00796 | integrin, alpha L (antigen CD11A (p180), | 4.9 | −1.4 | 1.7 |
| | | lymphocyte function | | | |
| 39286_at | D64109 | transducer of ERBB2, 2 | 1.9 | 3.0 | 3.0 |
| 40329_at | AL0312 | ring finger protein 1 | 1.3 | 3.2 | 1.7 |
| | 28 | | | | |
| 41743_i_at | AF0610 | tumor necrosis factor alpha-inducible cellular | 1.7 | 1.5 | 2.3 |
| | 34 | protein | | | |
| 34335_at | AI76553 | ephrin-B2 | 1.6 | 1.5 | 1.7 |
| | 3 | | | | |
| 34818_at | X96381 | ets variant gene 5 (ets-related molecule) | 2.5 | 1.5 | 2.0 |
| 40951_at | AL0492 | | 1.7 | 1.7 | 1.9 |
| | 50 | | | | |
| 292_s_at | L29219 | CDC-like kinase 1 | 1.1 | 2.5 | 1.7 |
| 31777_at | AF0064 | muscle, skeletal, receptor tyrosine kinase | 1.2 | −5.7 | −2.6 |
| | 64 | | | | |
| 33069_f_at | U06641 | UDP glycosyltransferase 2 family, | −2.5 | 1.1 | −1.7 |
| | | polypeptide B15 | | | |
| 34068_f_at | X86174 | synovial sarcoma, X breakpoint 1 | −1.1 | −2.5 | −2.8 |
| 35081_at | D14838 | fibroblast growth factor 9 (glia-activating | −1.0 | −4.9 | −2.3 |
| | | factor) | | | |
| 35109_at | AB0182 | KIAA0756 protein | −5.3 | −1.0 | −2.6 |
| | 99 | | | | |
| 37871_at | X68830 | islet amyloid polypeptide | −3.0 | −0.9 | −2.0 |
| 40322_at | D12763 | interleukin 1 receptor-like 1 | −1.1 | −4.6 | −2.1 |
| 40387_at | U80811 | endothelial differentiation, lysophosphatidic | −4.3 | 1.4 | −3.5 |
| | | acid | | | |
| 32083_at | AF0278 | transmembrane 7 superfamily member 1 | −1.0 | −10.6 | −3.2 |
| | 26 | (upregulated in kidney) | | | |
| 35178_at | W27944 | Wnt inhibitory factor-1 | −6.5 | 1.1 | −5.3 |
| 39407_at | M22488 | bone morphogenetic protein 1 | −1.2 | −3.7 | −3.0 |
| 32834_r_at | AF0135 | sudD (suppressor of bimD6, Aspergillus | −3.7 | −1.1 | −3.0 |
| | 91 | nidulans) homolog | | | |
| 39448_r_at | W27095 | B7 protein | −1.9 | −1.3 | −1.6 |
| 41244_f_at | X80910 | protein phosphatase 1, catalytic subunit, beta | −2.6 | −1.7 | −2.1 |
| | | isoform | | | |
| 32531_at | X52947 | gap junction protein, alpha 1, 43kD (connexin | −1.2 | −5.7 | −3.2 |
| | | 43) | | | |
| 32583_at | J04111 | v-jun sarcoma virus 17 oncogene homolog | 2.0 | −16.0 | −3.7 |
| | | (avian) | | | |
| 1152_i_at | J00117 | chorionic gonadotropin, beta polypeptide | −2.8 | −2.3 | −6.1 |
| 618_at | M26167 | platelet factor 4 variant 1 | −2.1 | −4.3 | −4.0 |

*Only 27 of the 197 genes (13.7%) changed in a consistent manner by >50% after MP treatment of cell lines and primary cells in vivo.
**Negative numbers indicate a decrease and positive an increase in expression.

### Example 5

### Genes That Discriminated Treatment Response

The relation between changes in gene expression after treatment and clinical outcome was assessed in patients treated with LDMTX plus MP, because this was the largest group with sufficiently long clinical follow-up (median: 3.7 years, range: 2.9-6.4 years for those who remained in remission). Using a Cox proportional hazard regression model, with lineage as a covariate, 146 gene probe sets that were related to relapse (Table 5; P<0.05) were identified. Permutation analysis indicated that the smallest P-value was achieved with 87 probe sets (P=0.028), although statistical significance for discriminating outcome was achieved using 75 to 146 probe sets. Hierarchical clustering using the six genes with the highest discriminating power (the first six genes shown in the table) clearly separated the five patients who relapsed from the 11 patients who remain in complete remission.

TABLE 5
Genes significantly correlated to treatment outcome as identified by Cox
proportional hazard regression analysis*

| | | | Median FC | Median FC | |
|---|---|---|---|---|---|
| | | Median FC | Relapse | Relapse | Weight |
| Identifier | Gene Name | CCR** | B-lineage | T-lineage | by LDA |
| AF070554 | clone 24582 mRNA | −2.3 | 2.9 | 1.7 | 0.295 |
| X94630 | CD97 antigen | −1.1 | 1.6 | 1.5 | 0.207 |
| AB003791 | carbohydrate (keratan sulfate Gal-6) sulfotransferase 1 | −1.2 | −2.9 | −4.3 | 0.193 |
| W72239 | clone = IMAGE-345279 | 1.3 | −1.2 | 1.0 | 0.186 |
| U20982 | insulin-like growth factor binding protein 4 | −1.1 | 1.6 | 2.8 | 0.175 |
| M15169 | adrenergic, beta-2-, receptor, surface | 1.1 | −3.1 | −4.3 | 0.167 |
| U77664 | ribonuclease P (38kD) | −1.4 | 1.6 | 1.5 | 0.149 |
| AL050064 | hypothetical protein FLJ11220 | 1.1 | 1.6 | 1.7 | 0.144 |
| AF023466 | glycine-N-acyltransferase | 1.7 | −5.5 | −2.6 | 0.144 |
| AB026190 | Kelch motif containing protein | 1.1 | −3.6 | −2.1 | 0.136 |
| X00734 | tubulin, beta, 5 | −1.2 | 2.0 | 1.3 | 0.134 |
| L35546 | glutamate-cysteine ligase, modifier subunit | 1.7 | −2.6 | 1.1 | 0.134 |
| X96586 | neutral sphingomyelinase (N-SMase) activation | 1.5 | −1.1 | 1.1 | 0.127 |
| | associated factor | | | | |
| X76057 | mannose phosphate isomerase | −1.2 | 2.2 | 1.2 | 0.125 |
| AB016194 | ELK1, member of ETS oncogene family | −1.6 | 1.7 | −1.1 | 0.124 |
| W27466 | heterogeneous nuclear ribonucleoprotein D-like | −1.2 | −3.6 | −2.0 | 0.123 |
| AJ131186 | nuclear matrix protein NMP200 related to splicing factor | 1.1 | −3.0 | −1.6 | 0.123 |
| | PRP19 | | | | |
| AF038187 | CS box-containing WD protein | 1.0 | 2.4 | 1.6 | 0.120 |
| AF045229 | regulator of G-protein signalling 10 | −1.1 | 1.9 | 1.5 | 0.120 |
| M29551 | protein phosphatase 3, catalytic subunit, beta isoform | 1.1 | 1.5 | 1.2 | 0.114 |
| AL050289 | chromosome 6 open reading frame 5 | 1.1 | −1.1 | −1.1 | 0.113 |
| Z46376 | hexokinase 2 | 1.6 | −7.2 | −1.3 | 0.111 |
| AF052159 | clone 24416 mRNA | −1.1 | 3.0 | 1.3 | 0.111 |
| L36983 | dynamin 2 | 1.2 | −1.4 | 1.2 | 0.108 |
| AF011468 | serine/threonine kinase 15 | −1.1 | −13.5 | −1.4 | 0.104 |
| U41303 | small nuclear ribonucleoprotein polypeptide N | 1.1 | −1.5 | 1.1 | 0.103 |
| M34641 | fibroblast growth factor receptor 1 (fms-related tyrosine | −1.1 | −1.9 | −1.4 | 0.103 |
| | kinase 2) | | | | |
| M60278 | diphtheria toxin receptor (epidermal growth factor-like | 1.2 | −5.3 | 1.3 | 0.100 |
| | growth factor) | | | | |
| AF010313 | etoposide-induced mRNA | 1.6 | −1.1 | 1.4 | 0.100 |
| J02871 | cytochrome P450, subfamily IVB, polypeptide 1 | −1.6 | 1.1 | −1.1 | 0.100 |
| AF030227 | vav 1 oncogene | 1.0 | 1.7 | 1.1 | 0.097 |
| L02547 | cleavage stimulation factor, 3′ pre-RNA, subunit 1, | 1.3 | −2.2 | 1.1 | 0.097 |
| | 50kD | | | | |
| L01042 | TATA element modulatory factor 1 | −1.2 | 4.8 | 1.3 | 0.097 |
| AF049910 | transforming, acidic coiled-coil containing protein 1 | 1.5 | 1.2 | 1.3 | 0.096 |
| AF054185 | proteasome (prosome, macropain) subunit | 1.0 | −3.5 | −1.2 | 0.095 |
| Y11392 | chromosome 21 open reading frame 2 | −1.7 | 1.2 | −1.1 | 0.094 |
| M30894 | T cell receptor gamma locus | 1.4 | −7.0 | 1.2 | 0.094 |
| M60974 | growth arrest and DNA-damage-inducible, alpha | 1.4 | −3.6 | 1.4 | 0.091 |
| X16901 | general transcription factor IIF, polypeptide 2 | 1.2 | 3.7 | 1.3 | 0.091 |
| L37936 | Ts translation elongation factor, mitochondrial | −1.2 | 1.5 | −1.1 | 0.090 |
| L39211 | carnitine palmitoyltransferase I, liver | 1.0 | 3.0 | 1.9 | 0.089 |
| AJ010842 | XPA binding protein 1; putative ATP(GTP)-binding | −1.1 | −2.0 | −1.1 | 0.087 |
| | protein | | | | |
| U46461 | disheveled, dsh homolog 1 (Drosophila) | −1.5 | −5.3 | −1.4 | 0.085 |
| AL080062 | DKFZP564I122 protein | −1.2 | −4.8 | −1.4 | 0.084 |
| D11466 | phosphatidylinositol glycan | −1.2 | −1.7 | −1.3 | 0.084 |
| AI767675 | chymotrypsin-like | 1.4 | −2.8 | 1.1 | 0.083 |
| U26648 | syntaxin 5A | 1.0 | 4.0 | 1.1 | 0.081 |
| D82351 | RNA binding motif, single stranded interacting protein 1 | 1.3 | 7.0 | 1.4 | 0.080 |
| D86966 | KIAA0211 gene product | −1.1 | −1.4 | −1.1 | 0.079 |
| S76346 | AML1 = AML1 {alternatively spliced, exons 5 and b} | −1.1 | −3.6 | −1.5 | 0.078 |
| M63256 | cerebellar degeneration-related protein (62kD) | −1.3 | −5.3 | −1.2 | 0.078 |
| D21211 | protein tyrosine phosphatase (APO-1/CD95 associated | −1.1 | −6.5 | −1.1 | 0.076 |
| | phosphatase) | | | | |
| U08377 | splicing factor, arginine/serine-rich 8 | 1.0 | 2.5 | −1.1 | 0.076 |
| AL096751 | M-phase phosphoprotein 9 | 1.6 | −3.7 | 2.1 | 0.075 |
| AB007940 | KIAA0471 gene product | 1.3 | −1.7 | 1.5 | 0.075 |
| Z12173 | glucosamine (N-acetyl)-6-sulfatase | −1.1 | 2.5 | 1.1 | 0.074 |
| AF070606 | clone 24411 mRNA | 1.1 | −2.9 | 1.0 | 0.074 |
| S40369 | glutamate receptor, ionotropic, kainate 5 | 1.5 | −3.7 | 1.5 | 0.074 |
| D38293 | adaptor-related protein complex 3, mu 2 subunit | 2.6 | −1.6 | 1.7 | 0.071 |
| W28191 | 43d1 Homo sapiens cDNA | −1.4 | −4.4 | −1.1 | 0.070 |
| U21936 | solute carrier family 15 (oligopeptide transporter) | −1.7 | −9.2 | −3.0 | 0.070 |
| U80764 | EST clone 122887 mariner transposon Hsmar1 | 1.0 | −1.4 | −1.1 | 0.070 |
| | sequence | | | | |
| D13666 | osteoblast specific factor 2 (fasciclin I-like) | −1.1 | −5.3 | −1.6 | 0.070 |
| M28211 | RAB4, member RAS oncogene family | 1.1 | 4.9 | 1.2 | 0.069 |
| AB015633 | transmembrane protein 5 | 1.1 | −5.3 | 1.2 | 0.067 |
| S59184 | RYK receptor-like tyrosine kinase | 1.0 | −2.6 | −1.1 | 0.067 |
| U11863 | amiloride binding protein 1 (amine oxidase) | 1.1 | −3.9 | 1.2 | 0.067 |
| AB011151 | KIAA0579 protein | 1.1 | 2.1 | −2.3 | 0.064 |
| X56807 | desmocollin 2 | −1.3 | −8.0 | −1.1 | 0.063 |
| U64805 | breast cancer 1, early onset | 1.5 | −1.4 | 1.0 | 0.062 |
| X12534 | RAP2A, member of RAS oncogene family | −2.8 | −1.1 | −1.5 | 0.062 |
| U50535 | Human BRCA2 region, mRNA sequence CG006 | −1.1 | 1.5 | 1.1 | 0.062 |
| W28518 | 48a1 Homo sapiens cDNA | −1.7 | −3.2 | −1.9 | 0.061 |
| AB022918 | alpha2,3-sialyltransferase | 1.0 | −3.6 | 1.1 | 0.061 |
| Z46606 | HLTF gene for helicase-like transcription factor | 1.1 | 2.7 | −1.1 | 0.060 |
| U88964 | interferon stimulated gene (20kD) | 1.1 | 1.9 | −1.1 | 0.060 |
| AL050002 | cDNA DKFZp564O222 | 1.1 | −1.8 | −1.1 | 0.060 |
| AL080149 | bromodomain-containing 1 | 1.2 | −1.1 | 1.2 | 0.059 |
| X03363 | v-erb-b2 erythroblastic leukemia viral oncogene | −1.4 | −3.5 | −1.1 | 0.058 |
| M83667 | CCAAT/enhancer binding protein (C/EBP), delta | 1.1 | −2.9 | 1.1 | 0.058 |
| M21574 | platelet-derived growth factor receptor, alpha | −1.4 | 1.5 | −1.5 | 0.058 |
| | polypeptide | | | | |
| AF030424 | histone acetyltransferase 1 | 1.1 | 3.7 | 1.3 | 0.057 |
| M19507 | Myeloperoxidase | −1.3 | −2.1 | −1.2 | 0.057 |
| AF020043 | chondroitin sulfate proteoglycan 6 (bamacan) | 1.1 | −1.2 | 1.2 | 0.057 |
| AI765533 | ephrin-B2 | −1.4 | −6.1 | −1.5 | 0.056 |
| M31932 | Fc fragment of IgG, low affinity IIa, receptor for (CD32) | −1.4 | −3.7 | −1.5 | 0.056 |
| AW026535 | leptin receptor gene-related protein | −1.1 | −1.6 | −1.3 | 0.055 |
| AB011090 | Max-interacting protein | 1.0 | −1.5 | 1.1 | 0.055 |
| AB006631 | K1AA0293 protein | −1.3 | −8.0 | 2.5 | 0.054 |
| X95632 | abl-interactor 12 (SH3-containing protein) | −2.0 | −11.3 | −1.3 | 0.054 |
| D25216 | KIAA0014 gene product | −1.1 | −4.0 | 1.0 | 0.054 |
| L35263 | mitogen-activated protein kinase 14 | 1.0 | 1.6 | 1.1 | 0.054 |
| M36881 | lymphocyte-specific protein tyrosine kinase | 1.1 | −1.5 | 1.0 | 0.054 |
| M13194 | excision repair cross-complementing rodent repair | 1.1 | 2.1 | 1.0 | 0.053 |
| | deficiency | | | | |
| AF007150 | angiopoietin-like 2 | 1.0 | −4.1 | 1.1 | 0.053 |
| U63743 | kinesin-like 6 (mitotic centromere-associated kinesin) | 1.0 | −3.4 | 1.1 | 0.052 |
| AL049415 | a disintegrin and metalloproteinase domain 19 | 1.0 | −2.3 | 1.6 | 0.052 |
| Y00636 | CD58 antigen, (lymphocyte function-associated antigen | | | | |
| | 3) | 1.0 | −1.4 | 1.1 | 0.050 |
| U07809 | nuclear factor I/A | −1.3 | −3.1 | −1.7 | 0.048 |
| AF031824 | cystatin F (leukocystatin) | −1.1 | −3.0 | 1.3 | 0.048 |
| AB007915 | KIAA0446 gene product | 1.1 | −1.3 | 1.3 | 0.048 |
| D26121 | ZFM1 protein alternatively spliced product | 1.0 | −4.9 | 1.7 | 0.047 |
| X84908 | phosphorylase kinase, beta | 1.0 | 1.5 | 1.1 | 0.047 |
| AF054186 | eukaryotic translation elongation factor 1 epsilon 1 | 1.0 | 1.7 | −1.1 | 0.045 |
| U37547 | baculoviral IAP repeat-containing 2 | −1.1 | 1.9 | −1.2 | 0.045 |
| AL120559 | cyclic AMP phosphoprotein, 19 kD | −1.3 | −2.0 | −1.2 | 0.045 |
| J03626 | uridine monophosphate synthetase | −1.1 | −2.1 | 1.2 | 0.043 |
| D38535 | inter-alpha (globulin) inhibitor H4 | −1.2 | −2.5 | 1.2 | 0.042 |
| M55210 | laminin, gamma 1 (formerly LAMB2) | 1.7 | −1.3 | 1.9 | 0.042 |
| AA808961 | proteasome (prosome, macropain) | 1.1 | 1.4 | 1.0 | 0.042 |
| D63789 | small inducible cytokine subfamily C, member 2 | 1.1 | −3.2 | −1.4 | 0.042 |
| D26361 | KIAA0042 gene product | −1.3 | 1.7 | −1.5 | 0.041 |
| AF052177 | KIAA1719 protein | −1.2 | −3.5 | 1.2 | 0.038 |
| AB023153 | MAK-related kinase | 1.1 | 3.0 | 1.0 | 0.038 |
| U77970 | neuronal PAS domain protein 2 | −1.1 | −5.5 | 1.3 | 0.038 |
| M96956 | teratocarcinoma-derived growth factor 1 | −1.3 | −10.9 | 1.1 | 0.036 |
| L17075 | activin A receptor type II-like 1 | 1.3 | −5.5 | −1.3 | 0.036 |
| U69127 | far upstream element (FUSE) binding protein 3 | −1.1 | 1.4 | −1.4 | 0.032 |
| D87457 | engulfment and cell motility 1 (ced-12 homolog, C. | −1.1 | 1.7 | −1.3 | 0.029 |
| | elegans) | | | | |
| AL050367 | cDNA DKFZp564A026 | 1.3 | −1.3 | 1.5 | 0.024 |
| H23429 | wingless-type MMTV integration site family, member 4 | 1.3 | 2.8 | −1.4 | 0.023 |
| X99141 | keratin, hair, basic, 3 | 1.0 | −4.3 | 1.2 | 0.022 |
| AL021707 | KIAA0063 gene product | 1.1 | −1.2 | 1.3 | 0.022 |
| L42621 | lymphocyte antigen 9 | 1.0 | −1.4 | 1.6 | 0.022 |
| Y09008 | uracil-DNA glycosylase | 1.2 | 2.5 | −1.2 | 0.021 |
| Z69030 | protein phosphatase 2, regulatory subunit B (B56) | −1.1 | 1.4 | −1.1 | 0.020 |
| AF070623 | clone 24468 mRNA | −1.4 | −5.1 | 2.0 | 0.020 |
| AB007923 | phosphodiesterase 4D interacting protein | 1.6 | −4.3 | 2.1 | 0.020 |
| M27878 | zinc finger protein 84 (HPF2) | 1.1 | 5.3 | −1.3 | 0.018 |
| U42360 | Putative prostate cancer tumor suppressor | −2.0 | −19.0 | 1.9 | 0.018 |
| M68891 | GATA binding protein 2 | −1.6 | −4.1 | 1.3 | 0.018 |
| AA883868 | ring finger protein 5 | −1.1 | −2.2 | 1.2 | 0.016 |
| L12535 | Ras suppressor protein 1 | −1.5 | −2.3 | 1.3 | 0.016 |
| D83664 | S100 calcium binding protein A12 (calgranulin C) | −1.7 | −7.2 | 1.1 | 0.016 |
| J04162 | Fc fragment of IgG, low affinity IIIb, receptor for (CD16) | 1.1 | −5.1 | 3.2 | 0.015 |
| AL080209 | hypothetical protein DKFZp586F2423 | 1.1 | −1.6 | 1.4 | 0.014 |
| M20137 | interleukin 3 (colony-stimulating factor, multiple) | −1.4 | −4.0 | −1.1 | 0.011 |
| W26981 | solute carrier family 17 | −2.0 | −2.7 | 1.1 | 0.010 |
| AF071771 | Zinc finger protein 143 (clone pHZ-1) | −1.3 | −4.0 | −0.5 | 0.009 |
| AB014562 | KIAA0662 gene product | 1.1 | 1.6 | −1.1 | 0.005 |
| L41162 | collagen, type IX, alpha 3 | −2.0 | −5.9 | 2.5 | 0.004 |
| X66436 | H. sapiens hsr1 mRNA (partial) | 1.1 | 1.6 | −1.4 | 0.004 |
| U17032 | Rho GTPase activating protein 5 | −1.3 | 3.2 | −4.9 | 0.003 |
| M60094 | H1 histone family, member T (testis-specific) | −2.3 | −7.5 | 1.9 | 0.002 |
| Z50115 | thimet oligopeptidase 1 | 1.2 | 2.2 | −1.2 | 0.001 |
| L08237 | Omithine Aminotransferase-Like 3 | 1.1 | 4.4 | −1.5 | 0.001 |
| *The Cox proportional hazard regression model, with lineage and changes in gene expression as covariates, identified 146 genes as significantly (P < 0.05) related to treatment outcome, based on changes in gene expression after treatment. The first six genes listed on the chart are those with the highest weight, as determined by LDA of these 146 genes. The median fold-change among patients who remained in | | | | | |
| # continuous complete remission (Median FC CCR) and the median fold-change among patients who relapsed (Median FC Relapse) are shown for each gene, with minus (−) indicating genes that exhibited a decrease in expression, whereas a positive number indicates those genes that exhibited an increase in expression after treatment with LDMTX/MP. | | | | | |

***************************************************************************************

Various publications, patent applications and patents are cited herein, the disclosures of which are incorporated by reference in their entireties.

That which is claimed:

1. A method for identifying genes and their expression products as screening targets for drugs which may be used to improve treatment of a selected condition, the method comprising:
(a) determining the expression level of one or more genes in a first sample from a patient affected by the selected condition prior to treatment with the selected therapy;
(b) determining the expression level of said one or more genes in a second sample from said patient following said treatment with the selected therapy;
(c) for each of said one or more genes, comparing the expression level measured in step (a) with the expression level measured in step (b);
wherein a gene whose expression level is significantly increased or significantly decreased following treatment with the selected therapy is identified, along with its expression products, as a screening target for drugs which may be used to improve treatment of the selected condition with the selected therapy.

2. The method of claim 1 wherein said first sample and said second sample comprise cells affected by said selected therapy.

3. A method for identifying genes and their expression products as screening targets for inhibitors which may be used to improve treatment of a selected condition with a selected therapy comprising:
(a) determining the expression level of one or more genes in a first sample from a patient affected by from the selected condition prior to treatment with the selected therapy;
(b) determining the expression level of said one or more genes in a second sample from said patient following said treatment with the selected therapy;
(c) for each of said one or more genes, comparing the expression level measured in step (a) with the expression level measured in step (b) to identify genes whose expression level is significantly increased following treatment with the selected therapy;
(d) repeating steps (a), (b), and (c) for each patient in a population of patients affected by the selected condition;
(e) determining which patients responded favorably to the selected therapy and which patients did not respond favorably to the selected therapy; and
(f) comparing the genes whose expression level is significantly; increased following treatment with the selected therapy in patients who responded favorably to the selected therapy with the genes whose expression level is significantly increased following treatment with the selected therapy in patients who did not respond favorably to the selected therapy, to thereby identify genes for which a significant increase in expression level following treatment with the selected therapy is correlated with a failure to respond favorably to the selected therapy;
wherein a gene whose increase inexpression level is correlated with a failure to respond favorably to the selected therapy is identified, along with its expression products, as a screening target for inhibitors which may be used to improve treatment of said selected condition with said selected therapy.

4. The method of claim 3 wherein said first sample and said second sample comprise cells that are affected by said selected therapy.

5. A method for identifying genes and their expression products as screening targets for inhibitors which may be used to treat a selected condition, said method comprising:
(a) determining the expression level of one or more genes in a first sample from a patient affected by the selected condition prior to treatment with a selected therapy;
(b) determining the expression level of said one or more genes in a second sample from said patient following said treatment with the selected therapy;
(c) for each of said one or more genes, comparing the expression level measured in step (a) with the expression level measured in step (b) to identify genes whose expression level is significantly increased following treatment with the selected therapy;
(d) repeating steps (a)-(c) for each patient in a population of patients affected by the selected condition;
(e) determining which patients responded favorably to the selected therapy and which patients did not respond favorably to the selected therapy; and
(f) comparing the genes whose expression level is significantly increased following treatment with the selected therapy in patients who responded favorably to the selected therapy with the genes whose expression level increased following treatment with the selected therapy in patients who did not respond favorably to the selected therapy, to thereby identify genes for which a significant increase in expression level following treatment with the selected therapy is correlated with a failure to respond favorably to the selected therapy;
wherein a gene whose increase in expression level is correlated with a failure to respond favorably to the selected therapy is identified, along with its expression products, as a screening target for inhibitors which may be used to treat the selected condition.

6. The method of claim 5 wherein said first sample and said second sample comprise cells that are affected by said selected therapy.

7. A method for identifying genes and their expression products as screening targets for mimics or activators which may be used to improve treatment of a selected condition with a selected therapy comprising:
(a) determining the expression level of one or more genes in a first sample from a patient affected by the selected condition prior to treatment with the selected therapy;
(b) determining the expression level of said one or more genes in a second sample from said patient following said treatment with the selected therapy;
(c) for each of said one or more genes, comparing the expression level measured in step (a) with the expression level measured in step (b) to identify genes whose expression level is significantly decreased following treatment with the selected therapy;
(d) repeating steps (a)-(c) for each patient in a population of patients affected by the selected condition; and
(e) determining which patients responded favorably to the selected therapy and which patients did not respond favorably to the selected therapy; and
(f) comparing the genes whose expression level is significantly decreased following treatment with the selected therapy in patients who responded favorably to the selected therapy with the genes whose expression level is significantly decreased following treatment with the selected therapy in patients who did not respond favorably to the selected therapy, to thereby identify genes for which a decrease in expression level following treatment with the selected therapy is correlated with a failure to respond favorably to the selected therapy;
wherein a gene whose decrease in expression level is correlated with a failure to respond favorably to the selected therapy is identified, along with its expression products, as a screening target for mimics or activators which may be used to improve treatment of said selected condition with said selected therapy.

8. The method of claim 7 wherein said first sample and said second sample comprise cells that are affected by said selected therapy.

9. A method for identifying genes and their expression products as screening targets for mimics or activators which may be used to treat a selected condition, said method comprising:
(a) determining the expression level of one or more genes in a first sample from a patient affected by from the selected condition prior to treatment with the selected therapy;
(b) determining the expression level of said one or more genes in a second sample from said patient following said treatment with the selected therapy;
(c) for each of said one or more genes, comparing the expression level measured in step (a) with the expression level measured in step (b) to identify genes whose expression level is significantly decreased following treatment with the selected therapy;
(d) repeating steps (a)-(c) for each patient in a population of patients affected by the selected condition;
(e) determining which patients responded favorably to the selected therapy and which patients did not respond favorably to the selected therapy; and
(f) comparing the genes whose expression level is significantly decreased following treatment with the selected therapy in patients who responded favorably to the selected therapy with the genes whose expression level is significantly decreased following treatment with the selected therapy in patients who did not respond favorably to the selected therapy, to thereby identify genes for which a decrease in expression level following treatment with the selected therapy is correlated with a failure to respond favorably to the selected therapy;
wherein a gene whose decrease in expression level is correlated with a failure to respond favorably to the selected therapy is identified, along with its expression products, as a screening target for mimic or activators which may be used to treat the selected condition.

10. The method of claim 9 wherein said first sample and said second sample comprise cells that are affected by said selected therapy.

11. A method for identifying genes and their expression products as screening targets for modulators which may be used to improve treatment of a selected condition with a selected therapy comprising:
(a) determining the expression level of one or more genes in a first sample from a patient affected by the selected condition prior to treatment with the selected therapy;
(b) determining the expression level of said one or more genes in a second sample from said patient following said treatment with the selected therapy;
(c) for each of said one or more genes, comparing the expression level measured in step (a) with the expression level measured in step (b) to identify genes whose expression level is significantly changed following treatment with the selected therapy;
(d) repeating steps (a)-(c) for each patient in a population of patients affected by the selected condition; and
(e) determining which patients responded favorably to the selected therapy and which patients did not respond favorably to the selected therapy; and
(f) comparing the genes whose expression level is significantly changed following treatment with the selected therapy in patients who responded favorably to the selected therapy with the genes whose expression level is significantly changed following treatment with the selected therapy in patients who did not respond favorably to the selected therapy, to thereby identify genes whose expression levels changed in patients who responded favorably to the selected therapy but whose expression levels did not change in patients who did not respond favorably to the selected therapy;
wherein a gene whose expression level following the selected therapy is significantly different in patients who responded favorably compared to patients who did not respond favorably to the selected therapy is identified, along with its expression products, as a screening target for modulators which may be used to improve treatment of said selected condition with said selected therapy.

12. The method of claim 11 wherein said first sample and said second sample comprise cells that are affected by said selected therapy.

13. A method for predicting whether a first therapy will have increased therapeutic efficacy in a patient in comparison with a second therapy, said method comprising the steps of:
(a) determining the expression level of one or more genes in a first sample from a patient affected by the selected condition prior to treatment with the first therapy wherein increased expression of said one or more genes after treatment is correlated with a favorable response to treatment;
(b) determining the expression level of said one or more genes in a second sample from the patient of (a) following said treatment with the first therapy;
(c) for each of said one or more genes, comparing the expression level measured in step (a) with the expression level measured in step (b) to determine the change in the expression level of said genes following treatment with the first therapy;
(d) repeating steps (a), (b), and (c) for each patient in a population of patients affected by the selected condition and treated with the first therapy; and
(e) determining the expression level of said one or more genes in a first sample from a patient affected by the selected condition prior to treatment with the second therapy;
(f) determining the expression level of said one or more genes in a second sample from the patient of (e) following said treatment with the second therapy;
(g) for each of said one or more genes, comparing the expression level measured in step (e) with the expression level measured in step (f) to determine the change in the expression level of said genes following treatment with the selected therapy;
(h) repeating steps (e), (f), and (g) for each patient in a population of patients affected by the selected condition and treated with the second therapy; and
(i) for each of said one or more genes, comparing the change in expression level following treatment with the first therapy with the change in expression level following treatment with the second therapy to thereby determine whether the expression levels of said one or more genes show a greater increase in expression levels following treatment with said first therapy than following treatment with said second therapy;
wherein a greater increase in expression levels for one or more of said genes following treatment with the first therapy in comparison with the expression level for said one or more genes following treatment with the second therapy results in a prediction that the first therapy will have increased therapeutic efficacy in a patient in comparison with the second therapy.

14. The method of claim 13 wherein said first sample and said second sample comprise cells that are affected by at least one therapy selected from said first therapy and said second therapy.

15. A method for predicting whether a first therapy will have increased therapeutic efficacy in a patient in comparison with a second therapy, said method comprising the steps of:
(a) determining the expression level of one or more genes in a first sample from a patient affected by the selected condition prior to treatment with the first therapy, wherein decreased expression of said one or more genes after treatment is correlated with a favorable response in a patient to treatment;
(b) determining the expression level of said one or more genes in a second sample from the patient of (a) following said treatment with the first therapy;
(c) for each of said one or more genes, comparing the expression level measured in step (a) with the expression level measured in step (b) to determine the change in the expression level of said genes following treatment with the selected therapy;
(d) repeating steps (a), (b), and (c) for each patient in a population of patients affected by the selected condition; and
(e) determining the expression level of said one or more genes in a first sample from a patient affected by the selected condition prior to treatment with the second therapy;
(f) determining the expression level of said one or more genes in a second sample from the patient of (e) following said treatment with the second therapy;
(g) for each of said one or more genes, comparing the expression level measured in step (e) with the expression level measured in step (f) to determine the change in the expression level of said genes following treatment with the selected therapy;
(h) repeating steps (e), (f), and (g) for each patient in a population of patients affected by the selected condition; and
(i) for each of said one or more genes, comparing the change in expression level following treatment with the first therapy with the change in expression level following treatment with the second therapy to thereby determine whether the expression levels of said one or more genes show a greater decrease in expression levels following treatment with said first therapy than following treatment with said second therapy;
wherein a greater decrease in expression levels for one or more of said genes following treatment with the first therapy in comparison with the expression level for said one or more genes following treatment with the second therapy results in a prediction that the first therapy will have increased therapeutic efficacy in a patient in comparison with the second therapy.

16. The method of claim 15 wherein said first sample and said second sample comprise cells that are affected by at least one therapy selected from said first therapy and said second therapy.

17. A method for predicting whether a first therapy will have increased deleterious effects in a patient in comparison with a second therapy, said method comprising the steps of:
(a) determining the expression level of one or more genes in a first sample from a patient affected by the selected condition prior to treatment with the first therapy, wherein increased expression of said one or more genes after treatment is correlated with deleterious effects in a patient;
(b) determining the expression level of said one or more genes in a second sample from the patient of (a) following said treatment with the first therapy;
(c) for each of said one or more genes, comparing the expression level measured in step (a) with the expression level measured in step (b) to determine the change in the expression level of said genes following treatment with the selected therapy;
(d) repeating steps (a), (b), and (c) for each patient in a population of patients affected by the selected condition; and
(e) determining the expression level of said one or more genes in a first sample from a patient affected by the selected condition prior to treatment with the second therapy;
(f) determining the expression level of said one or more genes in a second sample from the patient of (e) following said treatment with the second therapy;
(g) for each of said one or more genes, comparing the expression level measured in step (e) with the expression level measured in step (f) to determine the change in the expression level of said genes following treatment with the selected therapy;
(h) repeating steps (e), (f), and (g) for each patient in a population of patients affected by the selected condition; and
(i) for each of said one or more genes, comparing the change in expression level following treatment with the first therapy with the change in expression level following treatment with the second therapy to thereby determine whether the expression levels of said one or more genes show a greater increase in expression levels following treatment with said first therapy than following treatment with said second therapy;
wherein a greater increase in expression levels for one or more of said genes following treatment with the first therapy in comparison with the expression level for said one or more genes following treatment with the second therapy results in a prediction that the first therapy will have increased deleterious effects in a patient in comparison with a second therapy.

18. The method of claim 17 wherein said first sample and said second sample comprise cells that are affected by at least one therapy selected from said first therapy and said second therapy.

19. A method for predicting whether a first will have increased deleterious in a patient in comparison with a second therapy, said method comprising the steps of:
(a) determining the expression level of one or more genes in a first sample from a patient affected by the selected condition prior to treatment with the first therapy, wherein decreased expression of said one or more genes after treatment is correlated with deleterious effects in a patient;
(b) determining the expression level of said one or more genes in a second sample from the patient of (a) following said treatment with the first therapy;
(c) for each of said one or more genes, comparing the expression level measured in step (a) with the expression level measured in step (b) to determine the change in the expression level of said genes following treatment with the selected therapy;
(d) repeating steps (a), (b), and (c) for each patient in a population of patients affected by the selected condition; and
(e) determining the expression level of said one or more genes in a first sample from a patient affected by the selected condition prior to treatment with the second therapy;
(f) determining the expression level of said one or more genes in a second sample from the patient of (e) following said treatment with the second therapy;
(g) for each of said one or more genes, comparing the expression level measured in step (e) with the expression level measured in step (f) to determine the change in the expression level of said genes following treatment with the selected therapy;
(h) repeating steps (e), (f), and (g) for each patient in a population of patients affected by the selected condition; and
(i) for each of said one or more genes, comparing the change in expression level following treatment with the first therapy with the change in expression level following treatment with the second therapy to thereby determine whether the expression levels of said one or more genes show a greater decrease in expression levels following treatment with said first therapy than following treatment with said second therapy;
wherein a greater decrease in expression levels for one or more of said genes following treatment with the first therapy in comparison with the expression level for said one or more genes following treatment with the second therapy results in a prediction that the first therapy will have increased deleterious effects in a patient in comparison with a second therapy.

20. The method of claim 19 wherein said first sample and said second sample comprise cells that are affected by at least one therapy selected from said first therapy and said second therapy.

21. The method of claim 13, wherein increased expression of said one or more genes after treatment is correlated with a favorable response in a patient to treatment with said first therapy.

22. The method of claim 13, wherein increased expression of said one or more genes after treatment is correlated with a favorable response in a patient to treatment with said second therapy.

23. The method of claim 15, wherein decreased expression of said one or more genes after treatment is correlated with a favorable response in a patient to treatment with said first therapy.

24. The method of claim 15, wherein decreased expression of said one or more genes after treatment is correlated with a favorable response in a patient to treatment with said second therapy.

25. The method of claim 17, wherein increased expression of said one or more genes after treatment is correlated with deleterious effects in a patient following treatment with said first therapy.

26. The method of claim 17, wherein increased expression of said one or more genes after treatment is correlated with deleterious effects in a patient following treatment with said second therapy.

27. The method of claim 19, wherein decreased expression of said one or more genes after treatment is correlated with deleterious effects in a patient following treatment with said first therapy.

28. The method of claim 19, wherein decreased expression of said one or more genes after treatment is correlated with deleterious effects in a patient following treatment with said second therapy.

29. The method of claim 21, wherein said one or more genes for which increased expression after therapy is correlated with a favorable response in a patient to treatment with said first therapy is identified by a method comprising:
(a) determining the expression level of one or more genes in a first sample from a patient affected by from the selected condition prior to treatment with said first therapy, wherein said first sample comprises cells that are known to be affected by said condition;
(b) determining the expression level of said one or more genes in a second sample from said patient following said treatment with said first therapy wherein said second sample comprises cells that are known to be affected by said condition;
(c) for each of said one or more genes, comparing the expression level measured in step (a) with the expression level measured in step (b) to identify genes whose expression level changed significantly following treatment with said first therapy;
(d) repeating steps (a)-(c) for each patient in a population of patients affected by the selected condition;
(e) determining which patients responded favorably to said first therapy and which patients did not respond favorably to said first therapy; and
(f) comparing the genes whose expression level increased significantly following treatment with said first therapy in patients who responded favorably to said first therapy with the genes whose expression level did not change significantly following treatment with said first therapy in patients who did not respond favorably to the said first therapy, to thereby identify genes for which an increase in expression following treatment with said first therapy is correlated with a favorable response in a patient to said first therapy.

30. The method of claim 22, wherein said one or more genes for which increased expression after therapy is correlated with a favorable response in a patient to treatment with said second therapy is identified by a method comprising:
(a) determining the expression level of one or more genes in a first sample from a patient affected by from the selected condition prior to treatment with said second therapy, wherein said first sample comprises cells that are known to be affected by said condition;
(b) determining the expression level of said one or more genes in a second sample from said patient following said treatment with said second therapy wherein said second sample comprises cells that are known to be affected by said condition;
(c) for each of said one or more genes, comparing the expression level measured in step (a) with the expression level measured in step (b) to identify genes whose expression level changed significantly following treatment with said second therapy;
(d) repeating steps (a)-(c) for each patient in a population of patients affected by the selected condition;
(e) determining which patients responded favorably to said second therapy and which patients did not respond favorably to said second therapy; and
(f) comparing the genes whose expression level increased significantly following treatment with said second therapy in patients who responded favorably to said second therapy with the genes whose expression level did not change significantly following treatment with said second therapy in patients who did not respond favorably to the said second therapy, to thereby identify genes for which an increase in expression following treatment with said second therapy is correlated with a favorable response in a patient to said second therapy.

31. The method of claim 23, wherein said one or more genes for which decreased expression after therapy is correlated with a favorable response in a patient to treatment with said first therapy is identified by a method comprising:
(a) determining the expression level of one or more genes in a first sample from a patient affected by from the selected condition prior to treatment with said first therapy, wherein said first sample comprises cells that are known to be affected by said condition;
(b) determining the expression level of said one or more genes in a second sample from said patient following said treatment with said first therapy wherein said second sample comprises cells that are known to be affected by said condition;
(c) for each of said one or more genes, comparing the expression level measured in step (a) with the expression level measured in step (b) to identify genes whose expression level changed significantly following treatment with said first therapy;
(d) repeating steps (a)-(c) for each patient in a population of patients affected by the selected condition;
(e) determining which patients responded favorably to said first therapy and which patients did not respond favorably to said first therapy; and
(f) comparing the genes whose expression level decreased significantly following treatment with said first therapy in patients who responded favorably to said first therapy with the genes whose expression level did not change significantly following treatment with said first therapy in patients who did not respond favorably to the said first therapy, to thereby identify genes for which an increase in expression following treatment with said first therapy is correlated with a favorable response in a patient to said first therapy.

32. The method of claim 24, wherein said one or more genes for which decreased expression after therapy is correlated with a favorable response in a patient to treatment with said second therapy is identified by a method comprising:
(a) determining the expression level of one or more genes in a first sample from a patient affected by from the selected condition prior to treatment with said second therapy, wherein said first sample comprises cells that are known to be affected by said condition;
(b) determining the expression level of said one or more genes in a second sample from said patient following said treatment with said second therapy wherein said second sample comprises cells that are known to be affected by said condition;
(c) for each of said one or more genes, comparing the expression level measured in step (a) with the expression level measured in step (b) to identify genes whose expression level changed significantly following treatment with said second therapy;
(d) repeating steps (a)-(c) for each patient in a population of patients affected by the selected condition;
(e) determining which patients responded favorably to said second therapy and which patients did not respond favorably to said second therapy; and
(f) comparing the genes whose expression level decreased significantly following treatment with said second therapy in patients who responded favorably to said second therapy with the genes whose expression level did not change significantly following treatment with said second therapy in patients who did not respond favorably to the said second therapy, to thereby identify genes for which an increase in expression following treatment with said second therapy is correlated with a favorable response in a patient to said second therapy.

33. The method of claim 25, wherein said one or more genes for which increased expression after therapy is correlated with deleterious effects in a patient in response to treatment with said first therapy is identified by a method comprising:
(a) determining the expression level of one or more genes in a first sample from a patient affected by from the selected condition prior to treatment with said first therapy, wherein said first sample comprises cells that are known to be affected by said condition;
(b) determining the expression level of said one or more genes in a second sample from said patient following said treatment with said first therapy wherein said second sample comprises cells that are known to be affected by said condition;
(c) for each of said one or more genes, comparing the expression level measured in step (a) with the expression level measured in step (b) to identify genes whose expression level changed significantly following treatment with said first therapy;
(d) repeating steps (a)-(c) for each patient in a population of patients affected by the selected condition;
(e) determining which patients responded favorably to said first therapy and which patients did not respond favorably to said first therapy; and
(f) comparing the genes whose expression level increased significantly following treatment with said first therapy in patients who responded favorably to said first therapy with the genes whose expression level did not change significantly following treatment with said first therapy in patients who did not respond favorably to the said first therapy, to thereby identify genes for which an increase in expression following treatment with said first therapy is correlated with deleterious effects in a patient to said first therapy.

34. The method of claim 26, wherein said one or more genes for which increased expression after therapy is correlated with deleterious effects in a patient in response to treatment with said second therapy is identified by a method comprising:
(a) determining the expression level of one or more genes in a first sample from a patient affected by from the selected condition prior to treatment with said second therapy, wherein said first sample comprises cells that are known to be affected by said condition;
(b) determining the expression level of said one or more genes in a second sample from said patient following said treatment with said second therapy wherein said second sample comprises cells that are known to be affected by said condition;
(c) for each of said one or more genes, comparing the expression level measured in step (a) with the expression level measured in step (b) to identify genes whose expression level changed significantly following treatment with said second therapy;
(d) repeating steps (a)-(c) for each patient in a population of patients affected by the selected condition;
(e) determining which patients responded favorably to said second therapy and which patients did not respond favorably to said second therapy; and
(f) comparing the genes whose expression level increased significantly following treatment with said second therapy in patients who responded favorably to said second therapy with the genes whose expression level did not change significantly following treatment with said second therapy in patients who did not respond favorably to the said second therapy, to thereby identify genes for which an increase in expression following treatment with said second therapy is correlated with deleterious effects in a patient to said second therapy.

35. The method of claim 27, wherein said one or more genes for which decreased expression after therapy is correlated with deleterious effects in a patient in response to treatment with said first therapy is identified by a method comprising:
(a) determining the expression level of one or more genes in a first sample from a patient affected by from the selected condition prior to treatment with said first therapy, wherein said first sample comprises cells that are known to be affected by said condition;
(b) determining the expression level of said one or more genes in a second sample from said patient following said treatment with said first therapy wherein said second sample comprises cells that are known to be affected by said condition;
(c) for each of said one or more genes, comparing the expression level measured in step (a) with the expression level measured in step (b) to identify genes whose expression level changed significantly following treatment with said first therapy;
(d) repeating steps (a)-(c) for each patient in a population of patients affected by the selected condition;
(e) determining which patients responded favorably to said first therapy and which patients did not respond favorably to said first therapy; and
(f) comparing the genes whose expression level decreased significantly following treatment with said first therapy in patients who responded favorably to said first therapy with the genes whose expression level did not change significantly following treatment with said first therapy in patients who did not respond favorably to the said first therapy, to thereby identify genes for which an increase in expression following treatment with said first therapy is correlated with deleterious effects in a patient to said first therapy.

36. The method of claim 28, wherein said one or more genes for which decreased expression after therapy is correlated with deleterious effects in a patient in response to treatment with said second therapy is identified by a method comprising:
(a) determining the expression level of one or more genes in a first sample from a patient affected by from the selected condition prior to treatment with said second therapy, wherein said first sample comprises cells that are known to be affected by said condition;
(b) determining the expression level of said one or more genes in a second sample from said patient following said treatment with said second therapy;
(c) for each of said one or more genes, comparing the expression level measured in step (a) with the expression level measured in step (b) to identify genes whose expression level changed significantly following treatment with said second therapy;
(d) repeating steps (a)-(c) for each patient in a population of patients affected by the selected condition;
(e) determining which patients responded favorably to said second therapy and which patients did not respond favorably to said second therapy; and
(f) comparing the genes whose expression level decreased significantly following treatment with said second therapy in patients who responded favorably to said second therapy with the genes whose expression level did not change significantly following treatment with said second therapy in patients who did not respond favorably to the said second therapy, to thereby identify genes for which an increase in expression following treatment with said second therapy is correlated with deleterious effects in a patient to said second therapy.

37. In a method of screening for modulators of a target to improve treatment of a selected condition, an improvement comprising the use of a target identified by the method of claim 1.

38. In a method of screening for modulators of a target to improve treatment of a selected condition, an improvement comprising the use of a target identified by the method of claim 3.

39. In a method of screening for modulators of a target to improve treatment of a selected condition, an improvement comprising the use of a target identified by the method of claim 5.

40. In a method of screening for modulators of a target to improve treatment of a selected condition, an improvement comprising the use of a target identified by the method of claim 7.

41. The method of claim 1 wherein said selected condition is cancer.

42. The method of claim 41 wherein said cancer is acute lymphoblastic leukemia (ALL).

43. The method of claim 42 wherein said selected therapy is selected from the group consisting of methotrexate and mercaptopurine.

44. The method of claim 43 wherein said selected therapy is selected from the group consisting of methotrexate (MTX; 1 gm/m²), mercaptopurine (MP; 1 gm/m²), MP with low dose MTX (180 mg//m²), and MP with high dose MTX (1 gm/m²).

45. A method of screening for drugs which may be used to improve treatment of acute lymphoblastic leukemia with a selected therapy comprising screening for modulators of a target gene selected from the group listed in Table 1 or an expression product of said target gene.

46. A method of screening for drugs which may be used to improve treatment of acute lymphoblastic leukemia comprising screening for inhibitors of a target gene selected from the group listed in Table 2B or an expression product of said target gene.

47. A method of screening for drugs which may be used to improve treatment of acute lymphoblastic leukemia comprising screening for mimics or activators of a target gene selected from the group listed in Table 2A or an expression product of said target gene.

48. A method of screening for drugs which may be used to improve treatment of acute lymphoblastic leukemia comprising screening for modulators of a target gene selected from the group listed in Table 5 or an expression product of said target gene.
